SEQUENCE LISTING (42 sequences)

SEQ ID NO: 1 - protein, 4 aa, Artificial Sequence
  Linker peptide
  misc_feature (2)..(2): Xaa can be any naturally occurring amino acid
GXSG

SEQ ID NO: 2 - protein, 4 aa, Artificial Sequence
  Linker peptide
GGSG

SEQ ID NO: 3 - protein, 4 aa, Artificial Sequence
  Linker peptide
GASG

SEQ ID NO: 4 - protein, 4 aa, Artificial Sequence
  Linker peptide
GSSG

SEQ ID NO: 5 - protein, 4 aa, Artificial Sequence
  Linker peptide
GTSG

SEQ ID NO: 6 - protein, 4 aa, Artificial Sequence
  Linker peptide
GNSG

SEQ ID NO: 7 - protein, 8 aa, Artificial Sequence
  Flag tag
DYKDDDDK

SEQ ID NO: 8 - protein, 18 aa, Mycoplasma bovis
DKKEEKKKVEEPAKQAEG

SEQ ID NO: 9 - protein, 20 aa, Mycoplasma bovis
EKEIKETSDSPKKDGEKVSD

SEQ ID NO: 10 - protein, 16 aa, Mycoplasma bovis
PVTTENNDSIKDVKSD

SEQ ID NO: 11 - protein, 18 aa, Mycoplasma bovis
DNTYLEQVKLDSVSDVIK

SEQ ID NO: 12 - protein, 15 aa, Mycoplasma bovis
AKKDYGFNSHFGEDF

SEQ ID NO: 13 - protein, 15 aa, Mycoplasma bovis
VSDFIAGAFQGIIDW

SEQ ID NO: 14 - protein, 15 aa, Mycoplasma bovis
VSDFIAGTFQGIIDW

SEQ ID NO: 15 - protein, 18 aa, Mycoplasma bovis
RELALDEYSLRIEYSASE

SEQ ID NO: 16 - protein, 15 aa, Mycoplasma bovis
IVTNDFILIGGDGYK

SEQ ID NO: 17 - protein, 15 aa, Mycoplasma bovis
KAKSVFEGGDILESY

SEQ ID NO: 18 - protein, 21 aa, Mycoplasma bovis
NETKEPKKEPEMDAPIAPPTD

SEQ ID NO: 19 - protein, 15 aa, Mycoplasma bovis
GEGQNNGKPGEDKPN

SEQ ID NO: 20 - protein, 21 aa, Mycoplasma bovis
GTNTNNNNTPMPNENATPSPN

SEQ ID NO: 21 - protein, 15 aa, Mycoplasma bovis
DVEEFMDFAVIEIDF

SEQ ID NO: 22 - protein, 18 aa, Mycoplasma bovis
YSKIDRPIILDTKKKEEV

SEQ ID NO: 23 - protein, 15 aa, Mycoplasma bovis
GDYFLENYIDEDQQK

SEQ ID NO: 24 - protein, 15 aa, Mycoplasma bovis
KAKNDQPLEDFDKEI

SEQ ID NO: 25 - protein, 15 aa, Mycoplasma bovis
DKFDVNVSSFIDDSF

SEQ ID NO: 26 - protein, 18 aa, Mycoplasma bovis
NTEPGKNPGGNTEPGKNP

SEQ ID NO: 27 - protein, 18 aa, Mycoplasma bovis
PETPKGPETPKGPETPKG

SEQ ID NO: 28 - protein, 18 aa, Mycoplasma bovis
PEAPKGPEAPKGPEAPKG

SEQ ID NO: 29 - protein, 16 aa, Mycoplasma bovis
KNPGGDKNPGGDKNPG

SEQ ID NO: 30 - protein, 15 aa, Mycoplasma bovis
GENTEPDKNPGGDKN

SEQ ID NO: 31 - protein, 209 aa, Artificial Sequence
  Epitope construct 1a
GGSGDKKEEKKKVEEPAKQAEGGASGEKEIKETSDSPKKDGEKVSDGSSGPVTTENNDSIKDVKSDGTSGDNTYLEQVKLDSVSDVIKGNSGAKKDYGFNSHFGEDFGGSGVSDFIAGTFQGIIDWGASGVSDFIAGAFQGIIDWGSSGRELALDEYSLRIEYSASEGTSGIVTNDFILIGGDGYKGNSGKAKSVFEGGDILESYGGSG

SEQ ID NO: 32 - protein, 209 aa, Artificial Sequence
  Epitope construct 1b
GGSGAKKDYGFNSHFGEDFGASGVSDFIAGTFQGIIDWGSSGVSDFIAGAFQGIIDWGTSGIVTNDFILIGGDGYKGNSGKAKSVFEGGDILESYGGSGRELALDEYSLRIEYSASEGASGDKKEEKKKVEEPAKQAEGGSSGEKEIKETSDSPKKDGEKVSDGTSGDNTYLEQVKLDSVSDVIKGNSGPVTTENNDSIKDVKSDGGSG

SEQ ID NO: 33 - protein, 171 aa, Artificial Sequence
  Epitope construct 2a
GGSGNETKEPKKEPEMDAPIAPPTDGASGGTNTNNNNTPMPNENATPSPNGSSGGEGQNNGKPGEDKPNGTSGDVEEFMDFAVIEIDFGNSGYSKIDRPIILDTKKKEEVGGSGGDYFLENYIDEDQQKGASGKAKNDQPLEDFDKEIGSSGDKFDVNVSSFIDDSFGGSG

SEQ ID NO: 34 - protein, 171 aa, Artificial Sequence
  Epitope construct 2b
GGSGKAKNDQPLEDFDKEIGASGDKFDVNVSSFIDDSFGSSGDVEEFMDFAVIEIDFGTSGYSKIDRPIILDTKKKEEVGNSGGDYFLENYIDEDQQKGGSGGEGQNNGKPGEDKPNGASGGTNTNNNNTPMPNENATPSPNGSSGNETKEPKKEPEMDAPIAPPTDGGSG

SEQ ID NO: 35 - protein, 109 aa, Artificial Sequence
  Epitope construct 3
GGSGKNPGGDKNPGGDKNPGGASGGENTEPDKNPGGDKNGSSGPETPKGPETPKGPETPKGGTSGPEAPKGPEAPKGPEAPKGGNSGNTEPGKNPGGNTEPGKNPGGSG

SEQ ID NO: 36 - DNA, 627 bp, Artificial Sequence
  Nucleic acid for epitope construct 1a
ggtggtagtggtgataaaaaagaagaaaaaaaaaaagtcgaagaacccgccaaacaagccgaaggtggtgcctcaggtgaaaaagaaattaaagaaacctcagattcacccaaaaaagatggtgaaaaagtctcagatggttcatcaggtcccgtcaccaccgaaaataatgattcaattaaagatgtcaaatcagatggtacctcaggtgataatacctacttagaacaagtcaaattagattcagtctcagatgtcattaaaggtaattcaggtgccaaaaaagattacggtttcaattcacacttcggtgaagatttcggtggttcaggtgtctcagatttcattgccggtacattccaaggtatcattgattgaggtgcctcaggcgtctccgatttcattgccggtgccttccaaggtattattgattgaggttctagcggtagagaattagccttagatgaatactcattaagaattgaatactcagcctcagaaggtacatcaggtattgtcaccaatgacttcattttaattggtggcgatggttacaaaggcaactcaggtaaagccaaatcagtcttcgaaggtggtgatattttagaatcatacggtggttcgggt

SEQ ID NO: 37 - DNA, 627 bp, Artificial Sequence
  Nucleic acid for epitope construct 1b
ggtggtagtggtgccaaaaaagattacggtttcaattcacacttcggtgaagatttcggtgcctcaggtgtctcagatttcattgccggtaccttccaaggtattattgattgaggttcatcaggtgtctcagatttcattgccggtgccttccaaggtattattgattgaggtacctcaggtatcgtcacaaatgatttcattttaattggtggtgacggttacaaaggcaattcaggtaaagccaaatcagtcttcgaaggtggtgatattttagaatcatacggtggttcaggtagagaattagccttagatgaatactcattaagaattgaatactcagcctcagaaggtgcctcaggtgataaaaaagaagaaaaaaaaaaagtcgaagaacccgccaaacaagccgaaggtggttcatcaggtgaaaaagaaattaaagaaacctcagattcacccaaaaaagatggtgaaaaagtctcagatggtacctcaggtgataatacctacttagaacaagtcaaattagattcagtctcagatgtcattaaaggtaactcaggtcccgtcaccaccgaaaataatgattcaattaaagatgtcaaatcagatggtggttcaggt

SEQ ID NO: 38 - DNA, 513 bp, Artificial Sequence
  Nucleic acid for epitope construct 2a
ggtggtagtggcaatgaaaccaaagaacccaaaaaagaacccgaaatggatgcccccattgccccccccaccgatggtgcctcaggtggtaccaataccaataataataatacccccatgcccaatgaaaatgccaccccctcacccaatggttcatcaggtggtgaaggtcaaaataatggtaaacccggtgaagataaacccaatggtacctcaggtgatgtcgaagaattcatggatttcgccgtcattgaaattgatttcggtaattcaggttactcaaaaattgatagacccattattttagataccaaaaaaaaagaagaagtcggtggttcaggtggtgattacttcttagaaaattacattgatgaagatcaacaaaaaggtgccagcggtaaagccaaaaatgatcaacccttagaagatttcgataaagaaattggttctagcggtgataaattcgatgtcaatgtctcatcattcattgatgattcattcggtggttcgggt

SEQ ID NO: 39 - DNA, 513 bp, Artificial Sequence
  Nucleic acid for epitope construct 2b
ggtggtagtggtaaagccaaaaatgatcaacccttagaagatttcgataaagaaattggtgcctcaggtgataaattcgacgtcaatgtctcctccttcattgacgattcattcggttcatcaggtgatgtcgaagaattcatggacttcgccgtcattgaaattgatttcggtacctcaggttactcaaaaattgatagacccattattttagataccaaaaaaaaagaagaagtcggtaattcaggtggtgattacttcttagaaaattacattgatgaagatcaacaaaaaggtggttcaggtggtgaaggtcaaaataatggtaaacccggtgaagataaacccaatggtgcctctggtggtaccaataccaataataataatacccccatgcccaatgaaaatgccaccccctcacccaatggttctagcggtaatgaaaccaaagaacccaaaaaagaacccgaaatggatgcccccattgccccccccaccgatggtggttcgggt

SEQ ID NO: 40 - DNA, 327 bp, Artificial Sequence
  Nucleic acid for epitope construct 3
ggtggtagtggtaaaaatcccggtggtgataaaaatcccggtggtgataaaaatcccggtggtgcctcaggtggtgaaaataccgaacccgataaaaatcccggtggtgataaaaatggttcatcaggtcccgaaacccccaaaggtcccgaaacccccaaaggtcccgaaacccccaaaggtggtacctcaggtcccgaagcccccaaaggtcccgaagcccccaaaggtcccgaagcccccaaaggtggtaattcaggtaataccgaacccggtaaaaatcccggcggtaatacagagcccggtaaaaatcccggtggttcgggt

SEQ ID NO: 41 - protein, 5 aa, Mycoplasma bovis
TPGEN

SEQ ID NO: 42 - protein, 4 aa, Mycoplasma bovis
SKKS

(19) United States
(12) Patent Application Publication
(10) Pub. No.: US 2024/0033337 A1
(43) Pub. Date: Feb. 1, 2024

(54) VACCINE FOR MYCOPLASMA BOVIS

(71) Applicants: INTERVET INC., Rahway, NJ (US); ATG: BIOSYNTHETICS GMBH, Merzhausen (DE)

(72) Inventors: Johanna Jacoba Elisabeth BIJLSMA, Nijmegen (NL); Tjerko KAMMINGA, Nijmegen (NL); Josef MAIER, Oberndorf am Neckar (DE)

(73) Assignees: INTERVET INC., Rahway, NJ (US); ATG: BIOSYNTHETICS GMBH, Merzhausen (DE)

(21) Appl. No.: 18/264,330

(22) PCT Filed: Feb. 10, 2022
(86) PCT No.: PCT/EP2022/053188

§ 371 Date: Aug. 4, 2023

(30) Foreign Application Priority Data
Feb. 11, 2021 (EP) 21156503.1

(51) Int. Cl. A61K 39/02; C07K 14/30; A61P 31/04

(52) U.S. Cl. CPC A61K 39/0241; C07K 14/30; A61P 31/04

(57) ABSTRACT

Currently, there is no effective vaccination against M. bovis on the market, and treatment options become increasingly limited due to restrictions in the use of, and resistance to antibiotics. This is complicated by results that demonstrate the induction of vaccine-enhanced disease, upon the use of certain M. bovis proteins as a vaccine. Thus, there is an urgent need for an effective and safe M. bovis vaccine. A novel vaccine composition was found that comprises one or more recombinant proteins which (combined) contain one or more epitopes from each of a set of specific M. bovis proteins. Vaccines based on these recombinant proteins were found to be safe, and were effective in protecting ruminants against infection and disease resulting from a severe challenge infection with M. bovis, as was apparent from a strong reduction of lung damage and colonisation of the trachea.

The present invention relates to the field of vaccinology, specifically to veterinary vaccines against infection or disease caused by Mycoplasma bovis. In particular the invention regards a composition comprising recombinant protein, a recombinant vector or a host cell expressing said recombinant protein, a vaccine comprising said recombinant protein, vector or host cell, and various methods and uses regarding the recombinant protein, the vector and/or the host cell.

During the farming of ruminants such as buffalo, goats, sheep, deer, and most prominently: cattle, several diseases need to be dealt with. Especially diseases of the digestive-, reproductive-, and respiratory systems affect the well-being of the animals, as well as the economics of the operation. A wide variety of viral-, bacterial-, and parasite pathogens are known to play a role in ruminant disease, and many vaccines and therapeutics are being used to prevent or overcome infection and disease.

One of the pathogens for which vaccination has so far not been effective is Mycoplasma bovis. M. bovis related disease (Mycoplasmosis) mainly affects cattle, but other animals may be carriers of the bacterium. Main symptoms of disease are pneumonia, arthritis and mastitis, however several others may occur. M. bovis is a commensal but can cause disease as a primary pathogen, for example following stress-full conditions such as from handling, shipping, or mixing of groups. In addition M. bovis often acts as a secondary pathogen, especially in the ‘bovine respiratory disease complex’ together with viruses and other bacteria. Infection spreads rapidly through a herd, for example from carriers with subclinical infection. Reviews on M. bovis and the diseases it causes are e.g. in The Merck veterinary manual (2016, 11^{th} ed., Wiley publ., ISBN: 0911910611); and: Mycoplasma diseases of ruminants (Nicolas et al., 2009, CABI publ., ISBN: 0851990126).

Mycoplasma bacteria belong to the taxonomic class of the Mollicutes. These are the simplest and smallest self-replicating organisms known. Having so few genes, makes that Mollicutes bacteria heavily rely on resources from their environment, so when cultured in vitro they typically require very rich media to grow. Also, the lack of a cell wall makes them impervious to several antibiotics.

M. bovis has a circular double-stranded DNA genome of about 1 million base pairs, encoding about 800 proteins. Several fully annotated M. bovis genome sequences are publicly available, for example from the reference strain PG45 (Wise et al., 2010, Inf. and Imm., vol. 79, p. 982-983): GenBank® accession number NC_014760; strain JF4278: GenBank acc. nr. NZ_LT578453; strain Hubei-1: NC_015725.1; strain HB0801: NC_018077.1; strain CQ-W70: NZ_CP005933.1; and strain NM 2012: NZ_CP011348.1.

So far no clear correlation is known between specific proteins encoded by M. bovis and factors involved in virulence, pathology, or protective immune-response.

Many attempts to produce an M. bovis vaccine have been described: as a bacterin, as a live attenuated vaccine, and various kinds of subunit vaccines based on (parts of) one or more of the bacterial proteins

No live attenuated vaccines, e.g. as described in WO 2010/124154, or subunit vaccines e.g. as described in Prysliak & Perez-Casal (2016, Can. J. Microbiol., vol. 62, p. 492-504), are currently available on the market.

Some vaccines of the bacterin type have been licensed, e.g. Pulmo-Guard™ MpB (Boehringer Ingelheim), others are only provided as autogenous vaccines for local use. However, M. bovis bacterin vaccines are not used much in practice, as they were found to be of limited efficacy; see: Nicholas et al. (2002, Vaccine, vol. 20, p. 3569-3575).

Reviews on M. bovis vaccinology are: Perez-Casal et al. (2017, Vaccine, vol. 35, p. 2902-2907), and Calcutt et al. (2018, Transbound. Emerg. Dis., vol. 65, p. 91-109). Several problems have so far hindered the development of an effective M. bovis vaccine. One issue is the occurrence of vaccine-enhanced disease linked to the use of M. bovis vaccines and the antibodies generated, see Maunsell et al. (2011, J. Vet. Intern. Med., vol. 25, p. 772-783). Consequently, a pure humoral immune-response may not be the optimal vaccination response.

Another, and potentially related, problem is the ability of this bacterium to change its antigenic repertoire by switching and varying in the expression of its ‘variable surface lipoproteins’ (Vsp's). For a review see: Lysnyansky et al. (1999, J. of Bact., vol. 181, p. 5734-5741). This is a family of membrane-anchored proteins encoded by a set of genes with frequent on/off switching and genomic rearrangements of their repeat sequences. While the Vsp's are very antigenic, they do not seem to induce a protective immune response.

Sachse et al. (2000, Inf. & Imm., vol. 68, p. 680-687) describe the characterisation of a number of epitopes in Vsp proteins of M. bovis. Several of those affirm the conviction in this field that Vsp's are correlated with overreaction of the immune system. For example, in preparatory experiments by the present inventors, the epitope: TPGEN (SEQ ID NO: 41) described in Sachse, FIG. 3, of repeat sequence RA1, was found to strongly react with sera obtained from calves having high lung lesion scores after M. bovis infection. The same was found for the epitope: SKKS (SEQ ID NO: 42), labelled as a6 in Sachse FIG. 4.

So unfortunately, the main possibilities for control of M. bovis infection or disease currently available in ruminant farming and veterinary practice, are the separation of sick animals, treatment with antibiotics and anti-inflammatories, and the identification and culling of chronically infected shedders. The lack of an effective M. bovis vaccine so far, is one of the large unmet needs in cattle farming today, and is one of the main reasons why the use of antibiotics in cattle farming cannot readily be further reduced.

Consequently, there is an urgent and long-felt need for an effective vaccine against infection and/or disease caused by M. bovis.

It is therefore an object of the present invention to overcome a disadvantage in the prior art, and to accommodate to a need in the field by providing a vaccine against M. bovis that does not enhance disease but that is safe, and that can effectively reduce symptoms of infection and/or disease caused by M. bovis, such as lung lesions and colonisation of the respiratory tract.

Surprisingly it was found that this object can be met, and consequently one or more disadvantages of the prior art can be overcome, by providing a recombinant fusion protein, or a combination of such recombinant fusion proteins, which fusion protein, respectively combination of fusion proteins, comprises one or more epitopes from each one of a specific selection of M. bovis proteins.

The project leading to this invention has received funding from the European Union's Horizon 2020 research and innovation programme under grant agreement no. 634942. In this multi-year project (MycoSynVac) the inventors have identified proteins from M. bovis that had a number of specific properties in common such as being: highly expressed, expressed on the surface, and being recognised by sera from vaccinated cattle which had only low levels of lung lesions after challenge infection with M. bovis. This resulted in the identification of a unique set of 13 M. bovis proteins that fitted that profile. Next, epitopes from these proteins were used for expression in fusion in one or more recombinant proteins, which can now be used in an M. bovis vaccine.

Calves vaccinated with a composition comprising these recombinant proteins, and subsequently challenged intratracheally with a virulent M. bovis strain, did not show vaccine-enhanced disease. On the contrary, the vaccinates had remarkably few lung lesions after challenge. Also, levels obtained from re-isolation of challenge bacteria were indicative of much reduced levels of invasion and colonisation of the trachea. The addition of epitopes from 4 specifically selected Vsp proteins to the recombinant fusion protein(s) could further add to the level of protection induced.

A reduction of these symptoms of M. bovis infection and disease to this extent, the inventors had not been observed before, and surpassed all published results on M. bovis vaccination.

It is not known exactly how or why epitopes from this particular selection of M. bovis proteins were capable of providing such effective immuno-protection against a virulent M. bovis challenge. Although the inventors do not want to be bound by any theory or model that might explain these findings, they consider that at the basis for these advantageous findings was the combination of synthetic biology data from several strains of M. bovis, with the reactivity data of sera obtained from vaccinated-challenged animals. To their surprise this allowed the identification of the specific proteins of which epitopes allow the generation of a safe and effective M. bovis vaccine. These epitopes can now be used for the vaccination of a target in different ways: as a subunit, or via expression by a variety of recombinant vectors or a host cell.

This was not at all obvious from any disclosure in the prior art. While some of the M. bovis proteins from the set of 13 have previously been considered as potential vaccine candidate, e.g. the M. bovis P48 lipoprotein, no publications exist wherein one of these proteins, or epitopes thereof, are used in a vaccine that is as effective as the present invention, or is used in this particular combination with epitopes from specific other M. bovis proteins.

Therefore, in one aspect the invention relates to a composition comprising one or more recombinant proteins, characterised in that the recombinant protein or the combination of recombinant proteins comprise at least one epitope from each of the Mycoplasma bovis (M. bovis) proteins with the GenBank accession number: WP_014829937, WP_075271052, WP_013456547, SBO45938, WP_013455936, WP_075271207, WP_013954974, WP_013954588, WP_013456028, WP_013954511, WP_075271115, WP_013456252, and WP_041309176, or from a homologue of said M. bovis proteins.

The composition according to the invention may be in any form that is suitable for the invention, e.g. a liquid, a solid, a powder, etc. The liquid may be aqueous or oily; the solid may be frozen or freeze-dried. Typically the composition will start as being an aqueous liquid such as a physiological buffer comprising the recombinant protein or the combination of recombinant proteins for the invention, which aqueous liquid can subsequently be used, processed, formulated or otherwise modified into another form when appropriate.

The term “comprising” (as well as variations such as “comprises”, “comprise”, and “comprised”) as used herein, intends to refer to all elements, and in any possible combination conceivable for the invention, that are covered by or included in the text section, paragraph, claim, etc., in which this term is used, even if such elements or combinations are not explicitly recited; and not to the exclusion of any of such element(s) or combinations.

Therefore any such text section, paragraph, claim, etc., can therefore also relate to one or more embodiments wherein the term “comprises” (or its variants) is replaced by terms such as “consist of”, “consisting of”, or “consist essentially of”.

A “protein” refers to a molecular chain of amino acids. Included within the definition of protein for the invention, are polypeptides, peptides and oligopeptides.

A “recombinant protein” for the invention, is a protein of which the amino acid sequence does not match with a protein occurring in nature. Such a protein thus has a molecular make-up that is artificial, and man-made. The recombinant proteins of the invention are fusion proteins comprising one or more or all of the epitopes as indicated for the invention. Consequently, the epitopes can all be present in the same recombinant protein, or can be provided in more than one recombinant protein, so that the combination of recombinant proteins comprises all the indicated epitopes.

Such recombinant proteins can be constructed and expressed using a variety of molecular-biological tools and protein-expression techniques, e.g. by manipulation in vitro of their encoding genetic information by way of molecular cloning. These, and other techniques are explained in great detail in standard text-books like Sambrook & Russell: “Molecular cloning: a laboratory manual” (2001, Cold Spring Harbour Laboratory Press; ISBN: 0879695773); Ausubel et al., in: Current Protocols in Molecular Biology (J. Wiley and Sons Inc, NY, 2003, ISBN: 047150338X); and C. Dieffenbach & G. Dveksler: “PCR primers: a laboratory manual” (CSHL Press, ISBN 0879696540); and “PCR protocols”, by: J. Bartlett and D. Stirling (Humana press, ISBN: 0896036421).

An “epitope” is well-known to be an antigenic molecular structure, here: a linear amino acid sequence. To activate cells of the immune system, an epitope needs to be of sufficient size, either on its own, or by being connected to a carrier molecule. For the present invention, the size minimum can also be complied with by being comprised in fusion in a recombinant protein for the invention. Typically linear proteinaceous epitopes are from about 5 to about 30 amino acids in size.

For the invention, the term ‘about’ means that the value of the number or range that this term refers to, can vary by±5% around that indicated value.

For the invention, the recombinant protein or the combination of recombinant proteins comprise at least one epitope from each of the indicated M. bovis proteins; no special requirements apply for the division or arrangement of these epitopes in the recombinant protein(s), and they can be comprised in a specific order or not. Also they can be grouped or be divided, both within one recombinant protein or over several recombinant proteins, etc.

The skilled person is perfectly capable of selecting and optimising -when desired- the arrangement of the epitopes for the invention in a recombinant protein for the invention.

Identifying an epitope in the M. bovis proteins as indicated for the invention, is well within the capabilities of the skilled person, when starting from the information disclosed herein and knowing which M. bovis proteins to use. In addition, the focus can be on linear epitopes. Several tools are available to assist in that approach, using both in silico- and wet-lab based techniques.

“M. bovis” is well-known in the field of the invention, and such a bacterium has the characterising features of its taxonomic group, such as the morphologic, genomic, and biochemical characteristics, as well as the biological characteristics such as physiologic, immunologic, or pathologic behaviour.

The international reference strain of M. bovis is M. bovis strain PG45. This bacterium is available e.g. from ATCC® as strain ‘Donetta PG45’ under acc. nr. 25523, and its genome is provided in GenBank™ acc. nr. NC_014760, which also provides full annotation.

GenBank™ is a well-known public sequence database, which can be accessed online at: www.ncbi.nlm.nih.gov.

General information on M. bovis bacteria is available e.g. from reference handbooks as indicated herein. Samples of an M. bovis for use in the invention can be obtained from a variety of sources, e.g. as a field isolate from a ruminant in the wild or on a farm, or from various laboratories, (depository) institutions, or (veterinary) universities. M. bovis bacteria can be readily identified using routine serological-, biochemical-, or molecular-biological tools. In addition much sequence information on M. bovis is available digitally in public sequence databases such as NCBI's GenBank, UniProt, and EMBL's EBI. In addition, a specialised public database on Mollicutes genomics is accessible at www.MolliGen.org.

As is also known in the field, the classification of a micro-organism in a particular taxonomic group is based on the combined knowledge of its features. The invention therefore also includes variants of M. bovis that are sub-classified therefrom in any way, for instance as a subspecies, strain, isolate, genotype, subtype or subgroup, and the like.

Further, it will be apparent to a person skilled in the field of the invention that while a particular Mollicutes bacterium for the invention may currently be assigned to the species M. bovis, that is a taxonomic classification that could change in time as new insights can lead to reclassification into a new or different taxonomic group. For example, upon its isolation in 1962, M. bovis was initially named “M. agalactiae subspecies bovis”, and was upgraded to species rank in 1976. However, as such a name-change does not change the bacterium itself, or its antigenic repertoire, but only it's scientific name or classification, such re-classified bacteria remain within the scope of the invention.

An “M. bovis protein” for the invention is a protein as indicated herein by the accession number of its GenBank entry, or is a homologue of such an M. bovis protein. For information purposes, the trivial names of the M. bovis proteins from M. bovis strains such as JF4278, PG45, or Hubei-1, are provided in Table 1.

In the composition according to the invention anyone, or more, or all of the indicated M. bovis proteins of the invention can be replaced by its homologue.

As the skilled person will appreciate, the M. bovis proteins indicated for the invention may be derived from any bacterium of the species M. bovis as described above, including variants of that species. Also, the one or more epitopes from those proteins may be obtained from the same M. bovis isolate, or from different M. bovis isolates. Consequently, the M. bovis proteins for the invention can have the amino acid sequence as in the GenBank entry indicated for the invention, or can have an amino acid sequence that is homologous to that sequence.

A “homologue” of an M. bovis protein for the invention is a protein that has a limited number of changes in its amino acid sequence as compared to the protein of which it is the homologue. Such amino acid changes can e.g. result from amino acid sequence variability for a specific M. bovis protein between different isolates from M. bovis, and can for example result from conservative amino acid substitutions, in which one amino acid residue is replaced with an amino acid residue having a similar side chain. Families of amino acid residues having similar side chains are well-known in the art, and include for example: amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. alanine, valine, leucine, isoleucine, proline, phenylalanine, methionine, tryptophan), beta-branched side chains (e.g. threonine, valine, isoleucine) and aromatic side chains (e.g. tyrosine, phenylalanine, tryptophan, histidine).

For the invention, the ‘limited number of changes in its amino acid sequence’ in a homologue of an M. bovis protein as indicated for the invention, means the homologue has at least 70% amino acid sequence identity to the amino acid sequence of the indicated GenBank protein entry. Compare for example the amino acid sequence of the M. bovis DUF31 protein WP_075271115, which shares about 70% aa sequence identity with WP_136831755, a similar protein from another M. bovis isolate. Similarly, the M. bovis DUF285 protein WP_013954588 shares about 75% amino acid sequence identity with WP_075271215, a similar DUF285 protein from another M. bovis isolate.

For the invention the amino acid sequence homology is to be determined by alignment over the full length of the M. bovis protein for the invention, using the ‘blastp’ algorithm with standard parameters, from the NCBI BLAST™ suite of alignment software as is available online at: blast.ncbi.nlm.nih.gov/Blast.cgi.

Preferred homologue of an M. bovis protein for the invention is a protein from one of the M. bovis strains JF4278, PG45, Hubei-1, HB0801, CQ-W70, or NM_2012.

TABLE 1
Details of the M. bovis proteins for the invention.

| M. Bovis protein | |
|---|---|
| GenBank acc. nr. | M. bovis protein description |
| WP_014829937 | Variable surface lipoprotein with P30 pfam domain |
| WP_075271052 | Protein with SGNH/GDSL hydrolase family domain |
| WP_013456547 | ATP-dependent zinc metalloprotease FtsH |
| SBO45938 | PnrA lipoprotein |
| WP_013455936 | BMP family ABC transporter substrate-binding protein |
| WP_075271207 | Variable surface lipopr. with |
| | PBP2_NikA_DppA_OppA_like domain |
| WP_013954974 | Bifunctional metallophosphatase/5′-nucleotidase |
| WP_013954588 | DUF285 domain-containing protein |
| WP_013456028 | Protein with a SMC_prok_A domain |
| WP_013954511 | Protein with a SMC_prok_A domain |
| WP_075271115 | DUF31 family protein |
| WP_013456252 | P80/S41 peptidase family protein |
| WP_041309176 | Peptidase C1A family protein |

Details of embodiments and of further aspects of the invention will be described below.

In an embodiment of the composition according to the invention, a recombinant protein for the invention is an isolated protein. ‘Isolated’ for the invention refers to being taken from its natural environ, for example as in being harvested and/or purified in some way, and subsequently taken up into an appropriate composition and container.

In an embodiment, the composition according to the invention is characterised in that the epitopes are at least 6 amino acids in size, preferably at least 8, 10, 11, 12, 13, 14, 15 , 16, 17, 18, 19, 20 or even at least 21 amino acids in size, in this order of preference.

In an embodiment, the composition according to the invention is characterised in that the epitopes are less than 50 amino acids in size, preferably less than 40, 30, 28, 25, 24, 23, or even less than 22 amino acids in size, in this order of preference.

In an embodiment, the composition according to the invention is characterised in that the epitopes are from 10 to 25 amino acids in size; preferably from 15 to 21 amino acids in size.

For the invention, ranges of values inlcude the numbers indicating the ends of those ranges, and all values in-between.

In an embodiment, the composition according to the invention is characterised in that the recombinant protein or the combination of recombinant proteins comprise from 1 to 5 epitopes from each of the indicated M. bovis proteins or their homologues for the invention; preferably from 1 to 4, or even from 1 to 3 epitopes are comprised from each of the indicated M. bovis proteins or a homologue thereof.

In an embodiment of the composition according to the invention, a homologue of an M. bovis protein for the invention has at least 70% amino acid sequence identity to the aa sequence of the indicated GenBank entry. Preferably at least 72, 75, 77, 80, 82, 85, 87, 90, 91, 92 , 93, 94, 95, 96, 97, 98, or at least 99% amino acid sequence identity to the aa sequence of the indicated GenBank entry, in that order of preference.

In a search to further improve the efficacy of a vaccine comprising the composition according to the invention, the inventors came to further surprising results in regard to the common perception in this field that Vsp's distract the immune system and may even cause vaccine-enhanced disease. Surprisingly, four M. bovis Vsp proteins were identified that could safely be used to provide a further improvement of the efficacy of a vaccine for reducing infection or disease caused by M. bovis. One or more epitopes from each of these selected Vsp proteins were added to the epitopes from the M. bovis proteins indicated for the invention. This induced a further reduction of the median lung lesion scores (LLS) in animals that were vaccinated with these combined epitopes after challenge. Also this reduced considerably the variability in the LLS observed, resulting in a very consistent low LLS in all vaccinates of a treatment group after challenge. In addition, this considerably further reduced the amount of M. bovis challenge bacteria that could be re-isolated from the respiratory tract.

Therefore in an embodiment, the composition according to the invention is characterised in that the recombinant protein or the combination of recombinant proteins also comprises at least one epitope from each of the M. bovis Vsp proteins with the GenBank accession number: SBO46569, SBO46572, SBO46576, and SBO46580, or from a homologue of said M. bovis Vsp proteins.

In an embodiment, the composition according to the invention is characterised in that the recombinant protein or the combination of recombinant proteins comprise from 1 to 3 epitopes from each of the indicated M. bovis Vsp proteins or their homologues for the invention; preferably 1 or 2 epitopes are comprised from each of the indicated M. bovis Vsp proteins or a homologue thereof.

For the M. bovis Vsp proteins for the invention, the same considerations apply as described above for the M. bovis proteins. For example: as for the M. bovis proteins for the invention, the M. bovis Vsp proteins for the invention are proteins as indicated herein by the accession number of a GenBank entry, or are a homologue of said M. bovis Vsp proteins, as defined herein above.

In the composition according to the invention any one, or more, or all of the indicated M. bovis Vsp proteins of the invention can be replaced by its homologue.

Details of the M. bovis Vsp proteins for the invention are presented in Table 2.

TABLE 2
Details of the M. bovis Vsp proteins for the invention.

| M. Bovis Vsp protein | M. bovis Vsp protein |
|---|---|
| GenBank acc. nr. | description |
| SBO46569 | VspA |
| SBO46572 | VspE |
| SBO46576 | VspE-variant |
| SBO46580 | VspK |

To improve features of a recombinant protein for the invention, such as its expression, stability, purification, and/or its presentation to the immune system of a target, the recombinant protein may comprise one or more sequences that can assist with such improvements.

Therefore in an embodiment, the composition according to the invention is characterised in that the recombinant protein or the combination of recombinant proteins also comprises one or more additional sequences selected from: signal-, transmembrane-, anchor-, linker-, spacer-, marker-, and cleavage sequences.

All these additional sequences are well-known in the field of the invention and readily available. Consequently the skilled person is perfectly capable of selecting and applying one or more of such additional sequences when appropriate.

The additional sequences may be applied in different variations or combinations, and e.g. in the case of the use of more than one recombinant protein for the invention, the additional sequences applied may be the same or may be different for the fusion proteins.

A ‘signal’ sequence can assist in directing the expressed recombinant protein to the surface of a vector or of a host cell comprising the vector. Signal sequences are typically between 10 and 30 amino acids in size.

A transmembrane sequence can assist in the presentation of a recombinant protein for the invention at the surface of the vector or the host cell by integrating the protein into the lipid-bilayer of the outer membrane, e.g. the bacterial membrane, the viral envelope, or the host cell membrane.

An anchor sequence can provide for the attachment of a recombinant protein for the invention to the outer membrane of a vector or a host cell. An anchor sequence is typically a 22-25 aa hydrophobic alpha helix; a well-known anchor sequence is a GPI-anchor.

Preferably the signal-, transmembrane-, or anchor- sequence is selected in such a way that it is effective in the vector, or in the host cell comprising the vector; therefore the sequence is preferably selected from a surface-expressed protein from a virus or a bacterium in case these are used as vector, or from the cell type of the host cell. In a preferred embodiment where the vector is a bacterium, the sequence is derived from a surface-expressed protein from the same genus of bacterium.

A ‘linker’ sequence can serve to introduce some physical distance between subsections of a recombinant protein for the invention, for example between two epitopes. Alternatively a linker may serve to distance the combined epitopes from the surface-membrane of the vector or host cell that expresses the recombinant protein, when that confirmation was chosen. All these may improve the presentation of the epitopes to the immune system of a target.

In a preferred embodiment a recombinant protein for the invention comprises at least one linker sequence between at least two of the epitopes that it comprises. More preferably a recombinant protein for the invention comprises at least one linker sequence between each of the epitopes that it comprises. Preferably a single linker is comprised between the epitopes.

In an embodiment the linker sequence placed between epitopes is between 2 and 20 amino acids in size.

Commonly used linker sequences to connect epitopes in a recombinant protein for the invention are for example described in well-known handbooks; for a review see: Chen et al., 2013 (Adv. Drug Deliv. Rev., vol. 65, p. 1357-1369).

In an embodiment the linker sequence is rich in Glycine residues; such linkers are typically flexible structures, which benefits epitope mobility. Preferably the linker sequence has the general amino acid sequence: GXSG (SEQ ID NO: 1), whereby the amino acids are expressed in standard one-letter IUPAC code, and whereby X is any amino acid sequence except Proline or Histidine. More preferably X is a small amino acid selected from: A, C, D, G, N, S, T and V. Even more preferably the linker is one or more selected from: Linker 1: GGSG (SEQ ID NO: 2); Linker 2: GASG (SEQ ID NO: 3); Linker 3: GSSG (SEQ ID NO: 4); Linker 4: GTSG (SEQ ID NO: 5); and/or Linker 5: GNSG (SEQ ID NO: 6).

For the invention a particular linker sequence may also be used more than once in a recombinant protein for the invention, either in the same site in the protein or in different sites in the protein. Preferably the linker sequences are placed in-between the epitope sequences

A ‘spacer’ sequence can for example serve to increase the physical distance between the epitopes for the invention and the surface membrane of a vector or a host cell. For that purpose a spacer is preferably placed at or near the N- or the C-terminus of a recombinant protein for the invention. A spacer sequence for the invention is preferably longer than 10 amino acids, and can be derived e.g. from (the sequence of) a surface protein from a virus or a bacterium used as a vector, or from a host cell.

A ‘marker’ (or tag-) sequence can assist e.g. in the detection and quantification or in the purification of a recombinant protein for the invention. Well-known markers are: affinity tags such as a Maltose binding protein (MBP)- or Histidine (His)-tag; epitope tags such as Myc-, HA-, V5- or Flag-tag; or fluorescent protein tags such as a GFP or YFP, or a part thereof, are all well-known in the art. The marker can be used for detection and quantification purposes, e.g. using imaging or binding with specific antibodies, e.g. in an IFT or an ELISA. Purification can be done e.g. using affinity chromatography, e.g. using an antibody column; a His-tag can also be bound to a nickel column.

A His-tag typically has from 4 to 10 Histidines, preferably the His-tag is a 6x Histidine tag, i.e. having 6 histidines. A Flag-tag is preferably: DYKDDDDK (SEQ ID NO: 7).

A recombinant protein for the invention may comprise one or more marker sequences. The marker sequence can be placed at any site in a recombinant protein for the invention; preferably the marker sequence is placed at or near the N- or the C-terminus of the recombinant protein.

A ‘cleavage’ sequence can assist in the purification of a recombinant protein for the invention, e.g. by allowing the recombinant protein to be cut off from the surface of a vector or host cell, or by cutting off one or more of the additional amino acid sequences such as linkers or markers. Typically a cleavage signal will be the target for a peptidase, e.g. trypsin or endopeptidase, e.g. TEV protease, thrombin, or Factor Xa protease.

In an embodiment of the composition according to the invention, the recombinant protein or the combination of recombinant proteins comprises: 1 epitope from each of the M. bovis proteins WP_075271052, WP_013456547, WP_013455936, WP_075271207, WP_013954588, WP_013456028, WP_013954511, WP_013456252, and WP_041309176, or from a homologue of said M. bovis proteins; two epitopes from each of the M. bovis proteins WP_014829937, SBO45938, and WP_013954974, or from a homologue of said M. bovis proteins; and three epitopes from the M. bovis protein WP_075271115, or from a homologue of said M. bovis protein.

With regard to the epitopes to be used for the invention, from the M. bovis proteins for the invention or their homologues, details are described in Table 3, specifically: the region of the epitope in the M. bovis proteins or homologues for the invention; the preferred location of the epitope in the M. bovis proteins or homologues for the invention; the preferred amino acid sequence of the epitope; the SEQ ID number of that aa sequence; and an arbitrary number of the epitope for internal reference.

In an embodiment of the composition according to the invention, the epitopes from the indicated M. bovis proteins are derived from one or more specific regions of the M. bovis proteins as indicated in Table 3 in the column “Epitope region”, or from one or more such regions of a homologue of one or more of said M. bovis proteins.

In an embodiment of the composition according to the invention, the epitopes from the indicated M. bovis proteins, are derived from one or more specific locations of the M. bovis proteins as indicated by their starting- and ending amino acid numbers in Table 3 in the column “Epitope aa start-end”, or from one or more such locations of a homologue of said M. bovis proteins.

In an embodiment of the composition according to the invention, the epitopes from the indicated M. bovis proteins, or from a homologue of said M. bovis proteins, have an amino acid sequence as specified in Table 3 in the column “Epitope sequence”, or is a homologue of that aa sequence.

As homologs of an epitope from the indicated M. bovis proteins may have equal efficacy, therefore in an embodiment of the composition according to the invention, an epitope from an M. bovis protein for the invention, has at least 90% amino acid sequence identity to the full length of the aa sequence of that epitope, using the Blastp program with standard parameters. Preferred is an amino acid sequence identity of at least 91, 92, 93, 94, 95, 96, 97, 98, or even 99%, in that order of preference. For example, epitopes number 6 and 7 (SEQ ID NOs: 13 and 14) of Table 3 are homologs from each other in that they differ in one of 15 amino acids, and thus share 93% amino acid sequence identity.

In the composition according to the invention any one, or more, or all of the epitopes from the indicated M. bovis proteins of the invention can be replaced by its homologue.

In an embodiment of the composition according to the invention wherein the recombinant protein or the combination of recombinant proteins comprises epitopes from M. bovis Vsp proteins, the recombinant protein or the combination of recombinant proteins comprises: 1 epitope from each of the M. bovis Vsp proteins SBO46569, SBO46572, and SBO46576, or from a homologue of said M. bovis Vsp proteins; and two epitopes from the M. bovis Vsp protein SBO46580, or from a homologue of said M. bovis Vsp protein. A homologue of an M. bovis Vsp protein for the invention, is as defined for homologues of M. bovis proteins.

With regard to the epitopes to be used for the invention from the M. bovis Vsp proteins for the invention or their homologues, details are described in Table 4, specifically: the region of the epitope in the M. bovis Vsp proteins or homologues for the invention; the preferred location of the epitope in the M. bovis Vsp proteins or homologues for the invention; the preferred amino acid sequence of the epitope; the SEQ ID number of that aa sequence; and an arbitrary number of the epitope for internal reference.

In an embodiment of the composition according to the invention, the epitopes from the indicated M. bovis Vsp proteins are derived from one or more specific regions of the M. bovis Vsp proteins as indicated in Table 4 in the column “Epitope region”, or from one or more such regions of a homologue of said M. bovis Vsp proteins.

| SEQ | M. bovis | | Epitope | | |
|---|---|---|---|---|---|
| ID | protein | | aa | | Epitope |
| NO: | GenB acc. nr. | Epitope region | start-end | Epitope sequence | no. |
| 8 | WP_014829937 | In N-terminus | 30-47 | DKKEEKKKVEEPAKQAEG | 1 |
| 9 | | Central region | 78-97 | EKEIKETSDSPKKDGEKVSD | 2 |
| 10 | WP_075271052 | N-terminus before lipase domain | 55-70 | PVTTENNDSIKDVKSD | 3 |
| 11 | WP_013456547 | Between TM1 and TM2 | 59-76 | DNTYLEQVKLDSVSDVIK | 4 |
| 12 | SBO45938 | N-terminus of P48 major surface | 173-187 | AKKDYGFNSHFGEDF | 5 |
| | | lipoprotein domain | | | |
| 13 | | Middle of basic membrane lipoprotein | 434-448 | VSDFIAGAFQGIIDW | 6 |
| | | (BMP) domain | | | |
| 14 | WP_013455936 | Middle of BMP domain | 241-255 | VSDFIAGTFQGIIDW | 7 |
| 15 | WP_075271207 | N-terminus of central bacterial | 434-451 | RELALDEYSLRIEYSASE | 8 |
| | | extracellular solute-binding | | | |
| | | protein domain | | | |
| 16 | WP_013954974 | In end of C-terminal domain | 601-615 | IVTNDFILIGGDGYK | 9 |
| 17 | | After end of C-terminal domain | 625-639 | KAKSVFEGGDILESY | 10 |
| 18 | WP_013954588 | In N-terminus | 17-37 | NETKEPKKEPEMDAPIAPPTD | 11 |
| 19 | WP_013954511 | Conserved NNDKP repeat section | 134-148 | GEGQNNGKPGEDKPN | 13 |
| 20 | WP_013456028 | In N-terminus | 74-94 | GTNTNNNNTPMPNENATPSPN | 12 |
| 21 | WP_075271115 | Central in DUF31 domain | 541-555 | DVEEFMDFAVIEIDF | 14 |
| 22 | | C-terminus in DUF31 domain | 614-631 | YSKIDRPIILDTKKKEEV | 15 |
| 23 | | C-terminus in DUF31 domain, more | 650-664 | GDYFLENYIDEDQQK | 16 |
| | | C-terminal | | | |
| 24 | WP_013456252 | C-terminus of Mycoplasma lipoprotein | 701-715 | KAKNDQPLEDFDKEI | 17 |
| | | domain | | | |
| 25 | WP_041309176 | C-terminus of C1A papain domain | 505-519 | DKFDVNVSSFIDDSF | 18 |

TABLE 4
Details of epitopes for the invention from M. bovis Vsp proteins

| SEQ | M. bovis Vsp | | Epitope | | |
|---|---|---|---|---|---|
| ID | protein GenBank | | aa | | Epitope |
| NO: | acc. nr. | Epitope region | start-end | Epitope sequence | no. |
| 26 | SBO46569 | RN1 repeat region | 73-90 | NTEPGKNPGGNTEPGKNP | vsp1 |
| 27 | SBO46572 | RE1 repeat region | 59-76 | PETPKGPETPKGPETPKG | vsp2 |
| 28 | SBO46576 | RE1v repeat region | 59-76 | PEAPKGPEAPKGPEAPKG | vsp3 |
| 29 | SBO46580 | RK1 repeat region | 62-77 | KNPGGDKNPGGDKNPG | vsp4 |
| 30 | | RG2-RK1 repeat | 77-91 | GENTEPDKNPGGDKN | vsp5 |
| | | fusion | | | |

In an embodiment of the composition according to the invention, the epitopes from the indicated M. bovis Vsp proteins, are derived from one or more specific locations of the M. bovis Vsp proteins as indicated by their starting- and ending amino acid numbers in Table 4 in the column “Epitope aa start-end”, or from one or more such locations of a homologue of said M. bovis Vsp proteins.

In an embodiment of the composition according to the invention, the epitopes from the indicated M. bovis Vsp proteins, or from a homologue of said M. bovis Vsp proteins, have an amino acid sequence as specified in Table 4 in the column “Epitope sequence”, or of a homologue of that aa sequence.

In the composition according to the invention any one, or more, or all of the epitopes from the indicated M. bovis Vsp proteins of the invention can be replaced by its homologue.

In an embodiment of the composition according to the invention, the composition comprises a recombinant protein for the invention comprising the epitopes from each of SEQ ID NO: 8 through 25 or from a homologue of one or more of those epitopes. Preferably the recombinant protein comprises the epitopes from each of SEQ ID NO: 8 through 30, or from a homologue of those epitopes.

In an embodiment of the composition according to the invention, a homologue of an epitope from an M. bovis Vsp protein for the invention has at least 90% amino acid sequence identity to the full length of the aa sequence of that epitope, using the Blastp program with standard parameters. Preferred is an amino acid sequence identity of at least 91, 92, 93, 94, 95, 96, 97, 98, or even 99%, in that order of preference.

Alternatively the epitopes can be divided into subsets and be included into more than one recombinant protein for the invention.

In an embodiment, the composition according to the invention is characterised in that the recombinant protein or the combination of recombinant proteins comprises each of the epitopes from SEQ ID NO: 8 through 25, ora homologue of said epitopes.

In a preferred embodiment the composition according to the invention is characterised in that the recombinant protein or the combination of recombinant proteins also comprises each of the epitopes from SEQ ID NO: 26 through 30, ora homologue of said epitopes.

In an embodiment the composition according to the invention comprises a recombinant protein for the invention comprising the epitopes from each of SEQ ID NO: 8 through 17, or a homologue of said epitopes, and a recombinant protein comprising the epitopes from each of SEQ ID NO: 18 through 25, or a homologue of said epitopes.

In a preferred embodiment the composition also comprises a recombinant protein for the invention comprising the epitopes from each of SEQ ID NO: 26 through 30, or a homologue of said epitopes.

Also, much variation can be applied in the layout of the epitopes in the one or more recombinant proteins for the invention. This may adapt or improve immune response, stability, expression level, etc., and is well within the routine capabilities of the skilled person. For example, one or more linker sequences can be used, e.g. placed alternately in-between the epitope sequences.

Therefore in an embodiment of the composition according to the invention, the one or more recombinant proteins for the invention comprise the linker sequence of SEQ ID NO: 1. More preferably the one or more recombinant proteins for the invention comprise the linker sequence of one or more of SEQ D NO: 2 through 6, and are placed in-between the epitope sequences.

Further, the epitopes in the one or more recombinant proteins for the invention can be oriented in respect to each other in different ways. In addition, different versions of their relative orientations may be used, and these can also be used in combination so that in the resulting composition each epitope is represented more than once but in different contexts.

Therefore in an embodiment the composition according to the invention comprises a recombinant protein for the invention comprising the amino acid sequences of each of SEQ ID NO: 8 through 17 and each of SEQ ID NO: 2 through 6 in the following order from N- to C-terminus: SEQ ID NO: 2-8-3-9-4-10-5-11-6-12-2-14-2-13-4-15-5-16-6-17-2.

Written in another way, this is: link1-Ep1-link2-Ep2-link3-Ep3-link4-Ep4-link5-Ep5-link1-Ep7-link2-Ep6-link3-Ep8-link4-Ep9-link5-Ep10-link1, whereby “linkX” refers to the linker number, and “EpX” refers to the epitope number as indicated in Table 3.

In a preferred embodiment said recombinant protein comprises the amino acid sequence of SEQ ID NO: 31.

In an alternate embodiment the composition according to the invention comprises a recombinant protein for the invention comprising the amino acid sequences of each of SEQ ID NO: 8 through 17 and each of SEQ D NO: 2 through 6 in the following order from N- to C-terminus: 2-12-3-14-4-13-5-16-6-17-2-15-3-8-4-9-5-11-6-10-2.

Written in another way, this is: link1-Ep5-link2-Ep7-link3-Ep6-link4-Ep9-link5-Ep10-link1-Ep8-link2-Ep1-link3-Ep2-link4-Ep4-link5-Ep3-link1.

In a preferred embodiment said recombinant protein comprises the amino acid sequence of SEQ ID NO: 32.

In an embodiment the composition according to the invention comprises a recombinant protein for the invention comprising the amino acid sequences of each of SEQ ID NO: 18-25 and 2-6 in the following order from N- to C-terminus: SEQ ID NO: 2-18-3-19-4-20-5-21-6-22-2-23-3-24-4-25-2.

Written in another way, this is: link1-Ep11-link2-Ep12-link3-Ep13-link4-Ep14-link5-Ep15-link1-Ep16-link2-Ep17-link3-Ep18-link1.

In a preferred embodiment said recombinant protein comprises the amino acid sequence of SEQ ID NO: 33.

In an alternate embodiment the composition according to the invention comprises a recombinant protein for the invention comprising the amino acid sequences of each of SEQ ID NO: 18-25 and 2-6 in the following order from N- to C-terminus: SEQ ID NO: 2-24-3-25-4-21-5-22-6-23-2-20-3-19-4-18-2.

Written in another way, this is: link1-Ep17-link2-Ep18-link3-Ep14-link4-Ep15-link5-Ep16-link1-Ep13-link2-Ep12-link3-Ep11-link1.

In a preferred embodiment said recombinant protein comprises the amino acid sequence of SEQ ID NO: 34.

In an embodiment the composition according to the invention comprises a recombinant protein for the invention comprising the amino acid sequences of each of SEQ ID NO: 26 through 30 and each of SEQ D NO: 2 through 6 in the following order from N- to C-terminus: SEQ ID NO: 2-29-3-30-4-27-5-28-6-26-2.

Written in another way, this is: link1-Vsp4-link2-Vsp5-link3-Vsp2-link4-Vsp3-link5-Vsp1-link1. Whereby “VspX” refers to the epitope number as indicated in Table 4.

In a preferred embodiment said recombinant protein comprises the amino acid sequence of SEQ ID NO: 35.

In an embodiment the composition according to the invention comprises more than one recombinant protein for the invention, wherein the recombinant proteins comprise the amino acid sequence of one or both of SEQ ID NO: 31 and 32; and of one or both of SEQ ID NO: 33 and 34.

Preferably the composition comprises recombinant proteins comprising the amino acid sequence of each of SEQ ID NO: 31 through 34.

More preferably the composition comprises recombinant proteins comprising each of the amino acid sequences of SEQ ID NO: 31 through 35.

As indicated above, the recombinant protein or the combination of recombinant proteins for the invention can comprise further sequences such as e.g. marker sequences.

In an embodiment a recombinant protein for the invention comprises the amino acid sequence of a 6x His-tag or of a Flag tag (SEQ ID NO: 7). More preferably the marker sequence is placed at the C-terminus of the recombinant protein.

As described above, the recombinant protein(s) of the invention can be provided in a composition according to the invention. Alternatively the protein(s) can be expressed and delivered by way of a suitable vector.

Therefore in a further aspect the invention relates to recombinant vector capable of expressing the recombinant protein or the combination of recombinant proteins as defined for the invention, and wherein said vector is selected from a nucleic acid, a replicon particle (RP), a virus, and a bacterium.

A “vector” is well-known in the field of the invention as a molecular structure that carries the genetic information (a nucleic acid sequence) for encoding a heterologous protein, with appropriate signals to allow its expression under suitable conditions, either independently or via a host cell, and preferably in vitro.

For the invention ‘expression’ refers to the well-known principle of the expression of protein from genetic information by way of transcription and/or translation.

Many types and variants of such a vector are known and can be used for the invention, ranging from nucleic acid molecules like DNA or RNA, to more complex structures such as virus-like particles and replicon particles, up to replicating recombinant micro-organisms such as a virus or a bacterium.

Depending on the type of vector employed one or more expression signals may be provided, either in cis (i.e. provided by the recombinant vector itself) or in trans (i.e. provided from a separate source).

A “recombinant” vector for the invention, is a vector of which the genetic constitution does not fully match with that of its parental counterpart. Such a vector thus has a molecular make-up that was changed, typically by manipulation in vitro of its genetic information by way of molecular cloning, and recombinant protein expression techniques. The changes made can serve to provide for, to improve, or to adapt the expression, manipulation, purification, stability and/or the immunological behaviour of the vector and/or of the protein it expresses.

The skilled person is well equipped to select and combine the required signals into operational combinations to make the recombinant vector according to the invention “capable of expressing” the recombinant protein(s) for the invention under appropriate conditions. Such elements can assist with the construction and cloning, such as restriction enzyme recognition sites or PCR primers. In addition these can be selected from well-known expression-regulating elements such as a: promoter, stop codon, termination signal, polyadenylation signal, 7-methylguanosine (7 mG) cap structure, and an intron with functional splice donor- and -acceptor sites.

In an embodiment of the recombinant vector according to the invention, the vector can be ‘alive’ (i.e. replicative), or can be ‘dead’ (i.e. non-replicative) e.g. from having been inactivated. Several methods and materials are known for inactivating a replicative vector according to the invention, for example using chemical or physical means; physical means are e.g. heating, irradiation (U.V. light, gamma-rays), or very high pressure; chemical means are e.g. incubation with merthiolate, formalin, diethylamine, binary ethylenamine, beta propiolactone, benzalkonium chloride or glutaraldehyde.

In an embodiment of the recombinant vector according to the invention, the vector is a nucleic acid.

In an embodiment of the recombinant vector according to the invention wherein the vector is a nucleic acid, the nucleic acid is a DNA expression plasmid.

A DNA expression plasmid has the appropriate signals for expression of a heterologous gene that is inserted into the plasmid, under the operational control of a promoter that is active in a suitable host cell. The DNA plasmid can then be inserted into a host cell in vitro by some method of transfection, e.g. using a biochemical substance as carrier, by mechanical means, or by electroporation, and will commence the expression of the heterologous gene insert. Typically such expression will be transient, as the plasmid usually lacks signals for stable integration into the genome of the host cell; consequently such a plasmid will typically not transform or immortalise the host cell. All these materials and procedures are well known in the art and are described in handbooks. Such DNA expression plasmids are commercially available from a variety of suppliers, for example the plasmid series: pcDNA™, pCR3.1™, pCMV™, pFRT™, pVAX1™, pCI™, Nanoplasmid™, pCAGGS etc.

In a preferred embodiment the DNA expression plasmid is a plasmid of the pFRT (ThermoFisher) or pCAGGS plasmids (Niwa et al., 1991, Gene, vol. 108, p. 193-199).

In an embodiment the DNA plasmid can be administered to a host organism, e.g. an animal, as a DNA vaccine, and provide for expression in vivo, e.g. with the help of antigen presenting cells from the host. Alternatively a host cell comprising the DNA plasmid is administered to a host organism.

A DNA expression plasmid can comprise several features for regulation of expression, purification, etc. One possible signal is an antibiotic resistance gene, which can be used for phenotypic selection during the construction, cloning, and production process. However when intended for administration to an animal target, such antibiotic selection is not desired for fear of generating antibiotic resistance.

In a preferred embodiment of the recombinant vector according to the invention, wherein the vector is a nucleic acid, and the nucleic acid is a DNA expression plasmid, the plasmid does not contain an antibiotic resistance gene.

The recombinant vector according to the invention, in the form of a DNA expression plasmid, can be delivered to a host cell or target organism, where it will express a recombinant protein for the invention in the host cell. Delivery of the DNA plasmid can be in several ways, e.g. by mechanical or chemical means, as naked DNA, or encapsulated with an appropriate (nanoparticulate) carrier, such as a protein, polysaccharide, lipid or a polymer. Well-known examples of nucleic acid carriers are dendrimers, lipid nanoparticles, cationic polymers and protamine.

A special form of the recombinant vector according to the invention, as a DNA expression plasmid, is when the plasmid provides for the transcription and delivery of replicon RNA.

Therefore in an embodiment of the recombinant vector according to the invention, wherein the vector is a nucleic acid and the nucleic acid is a DNA expression plasmid, the plasmid provides for the transcription of a replicon RNA.

A “replicon RNA”, also known as: self-amplifying mRNA, is a self-replicating RNA which contains, in addition to the nucleic acid encoding a recombinant protein for the invention, elements necessary for RNA replication, such as a replicase gene. However, unlike a replicon particle (RP), a replicon RNA is not packaged by viral structural proteins, and is thus less efficient at entering host cells.

The replicon RNA-transcribing DNA plasmid can be delivered to a host cell in the same way as a protein-expressing plasmid.

Use of a DNA expression plasmid transcribing replicon RNA provides an advantage over use of a DNA expression plasmid expressing protein, because the replicon RNA provides for an amplification step in a host cell or in an animal target: the translation of the replicase gene makes the replicon RNA produce subgenomic messenger RNA encoding the recombinant protein for the invention. This results in the expression of high amounts of said protein in the host cell, respectively in the target.

In a preferred embodiment of the recombinant vector according to the invention, wherein the vector is a nucleic acid, the nucleic acid is a DNA expression plasmid, and the plasmid transcribes a replicon RNA: the replicon RNA is an Alphavirus-based replicon RNA; more preferably the Alphavirus-based replicon RNA is a Venezuelan equine encephalitis virus (VEEV) based replicon RNA.

An example of a DNA expression plasmid transcribing a VEEV replicon RNA is e.g. a pVAX plasmid (ThermoFisher), comprising VEEV non-structural protein genes 1-4, driven by a eukaryotic promoter such as a human CMV immediate early gene 1 promoter.

In an embodiment of the recombinant vector according to the invention wherein the vector is a nucleic acid, the nucleic acid is an RNA molecule.

The RNA molecule for the invention can have different forms and functions, for example can be an mRNA or can be a replicon RNA.

A recombinant vector according to the invention, as an RNA molecule can be delivered to a target host organism, or to a host cell in vitro, in different ways, e.g. by mechanical- or chemical means, or by encapsulation with an appropriate (nanoparticulate) carrier, such as a protein, polysaccharide, lipid or a polymer, as described above. To stabilise the RNA certain chemical modifications may be applied, e.g. to the ribonucleotides or to their backbone, or the incorporation of nucleotide-analogues, all well-known in the art.

In an embodiment of the recombinant vector according to the invention wherein the vector is a nucleic acid and the nucleic acid is an RNA molecule, the RNA molecule is an mRNA.

An “mRNA” is well-known in the art, and can be a prokaryotic mRNA or a eukaryotic mRNA. Eukaryotic mRNAs typically have a 5′ 7 mG cap and a 3′ poly-A tail. An mRNA can be delivered to a host organism, or to a host cell in vitro, by way of transfection and/or by using an appropriate carrier, e.g. a polymer or a cationic lipid.

In an embodiment of the recombinant vector according to the invention wherein the vector is a nucleic acid and the nucleic acid is an RNA molecule, the RNA molecule is a replicon RNA.

The replicon RNA can be produced in vitro e.g. using a pVAX plasmid construct, and then be administered to a host cell or a target organism, using any suitable method.

The recombinant vector according to the invention can also be delivered and expressed to a target in the form of a macro-molecular structure that resembles a virion. Examples are virus-like particle (VLPs) or replicon particles (RPs). Known as ‘single cycle’ infectious particles, these contain features necessary to infect a host cell, and express the heterologous gene it carries, encoding a recombinant protein for the invention. However, as a built-in safety feature, they will typically not be capable of full viral replication, for lack of relevant parts of the viral genome from which they were constructed.

“RPs” are well-known, and several RPs have been developed as a platform for the expression and delivery of a variety of proteins. Favourable basis for an RP is an Alphavirus, because of its broad host-range and rapid replication. Of course appropriate safety measures need to be taken to attenuate and control the infection of such RPs, as some Alphaviruses are highly pathogenic in their wildtype form. For a review, see: Kamrud et al., 2010, J. Gen. Virol., vol. 91, p. 1723-1727, and: Vander Veen, et al., 2012, Anim. Health Res. Rev., vol. 13, p. 1-9.

In an embodiment of the recombinant vector according to the invention wherein the vector is an RP, the RP is an Alphavirus RP.

Preferably the Alphavirus RP is a VEEV RP.

Alphavirus RPs based on VEEV have been applied as recombinant vector vaccine for human- and animal targets. Methods and tools to construct, test, and use VEEV-based Alphavirus RPs are well-known and available, see for example: Pushko et al., 1997, Virology, vol. 239, p. 389-401, and: WO 2019/110481. Preferred VEEV RP technology is the SirraVax™ RNA Particle technology (Harrisvaccine).

In an embodiment a pVAX plasmid construct is used to produce RPs: RNA is produced from the plasmid which is then transfected into the host cells together with helper RNAs encoding in trans the VEEV structural proteins.

Recombinant vectors for the expression and delivery of a heterologous antigen in the form of a live replicating recombinant micro-organism are well-known in the art. These provide for an efficient method of vaccination, as the live vector replicates in the target, which stimulates the target's immune system and amplifies the vector. Assembly and modification of a wide variety of replicating recombinant vectors is routine using standard molecular-biological techniques.

Many different species of micro-organism have been used over time as replicating recombinant vector, and for a variety of human- and animal targets.

In an embodiment of the recombinant vector according to the invention wherein the vector is a virus, the virus is selected from the group consisting of: a Herpesvirus, a Poxvirus, a Retrovirus, a Paramyxovirus, a Rhabdovirus, a Baculovirus and an Adenovirus.

For use in a ruminant target, the recombinant vector virus is preferably a Herpes virus, e.g. a bovine Herpes virus such as BoHV-1 (also known as infectious bovine rhinotracheitis virus), a pox virus such as a cowpox or a Vaccinia virus, a bovine Adenovirus, a bovine Paramyxovirus such as Bovine Parainfluenza virus 3, or a Rhabdovirus such as Rabies virus.

A recombinant Baculovirus vector can be used for the efficient expression of the recombinant protein(s) for the invention in insect cells.

In an embodiment of the recombinant vector according to the invention wherein the vector is a bacterium, the bacterium is selected from the group consisting of the genera Escherichia, Bacillus, Salmonella, Caulobacter, Lactobacillus, and Mycoplasma.

Bacterial vectors from all these genera have been described in the art for the expression of heterologous antigens in different ways. For example when in an attenuated form, the bacterial vector can be used as a live vector to infect a target host, and in this way deliver the heterologous antigen, here: the recombinant protein(s) for the invention. More often however, such bacterial vectors are used as effective expression vector when cultured in vitro. The culture can then be harvested and used for example in an inactivated form to vaccinate a target. Alternatively, the recombinant protein can be isolated from the (inactivated) culture and be used in a subunit vaccine.

In an embodiment of the recombinant vector according to the invention wherein the vector is a bacterium and the bacterium is a Mycoplasma, the Mycoplasma is M. pneumoniae (Mpneumo).

A minimal-genome version of M. pneumoniae, has been described by Lluch-Senar et al. (2015, Mol. Syst. Biol., vol. 11:780), and Trussart et al. (2017, Nature Communications, vol. 8, doi.org/10.1038/ncomms14665).

Different versions of a recombinant protein for the invention were cloned into an Mpneumo vector, cultured to sufficient density, harvested, inactivated, and used with an adjuvant to vaccinate calves. Details are described in the Examples.

Therefore in a preferred embodiment of the recombinant vector according to the invention wherein the vector is a bacterium and the bacterium is a Mycoplasma, the Mycoplasma is Mpneumo.

For the construction of a live recombinant vector, typically an expression cassette is inserted into a locus in the vector's genome. Different techniques are available to make that insertion, and to make a random or a directed insertion into the vector's genome. For example by using the appropriate flanking sections from the genome of the vector to direct the integration of the cassette by a homologous recombination process, e.g. by using overlapping cosmids. Alternatively the integration may be done by using a transposon insertion, or the CRISPR/Cas technology.

An ‘expression cassette’ is a nucleic acid fragment comprising a nucleotide sequence encoding a protein and a promoter to drive the transcription and enable the expression of the encoded protein. The termination of the transcription may be provided by sequences provided by the genomic insertion site of the cassette, or the expression cassette can itself comprise a termination signal, such as a transcription terminator. In an expression cassette, both the promoter and the terminator need to be in close proximity to the encoding nucleotide sequence of which they regulate the expression; this is termed being ‘operatively linked’.

As will be apparent to a skilled person, an expression cassette is a self-contained expression module, therefore its orientation in a vector's genome is generally not critical.

Having such an expression cassette makes the recombinant vector according to the invention ‘capable of expressing a recombinant protein’ for the invention. The cassette can be in the form of DNA or of RNA, to suit the different types of recombinant vectors according to the invention. For the invention many different forms and embodiments of the encoding nucleotide sequence in the expression cassette for the invention are possible.

Therefore, in an embodiment the recombinant vector according to the invention comprises a nucleotide sequence encoding the one or more recombinant proteins for the invention, whereby the recombinant protein(s) can be in any form or embodiment as outlined herein above, i.e. in single or in multiple recombinant proteins, with or without linker sequences, and with different sets of epitope sequences, in different orientations and combinations.

The nucleotide sequence encoding a recombinant protein for the invention can be based on a consensus sequence. As is well-known, to obtain such a consensus sequence, either amino acid- or encoding nucleotide sequences of the indicated proteins for the invention, or of their indicated epitopes, are compared between different strains of M. bovis, and a consensus sequence is derived from that comparison. Typically this is done by gathering sequence information from databases or from analysing isolates, and comparing their sequences using an appropriate computer program.

In a preferred embodiment a consensus sequence for the invention is a homologue as defined herein above.

When needed, the nucleotide sequence encoding the one or more recombinant proteins for the invention can be adapted and fine-tuned to optimise expression and/or to adapt or improve features of the recombinant protein(s). Therefore the nucleotide sequence may also encode one or more additional sequences such as signal-, transmembrane-, anchor-, linker-, marker-, or cleavage sequences, all as described herein above.

In addition, to optimise the encoding nucleotide sequence for expression in a specific type or species of recombinant vector, host cell, or target organism, the encoding nucleotide sequence may be subjected to codon optimisation. This is well-known in the art and is commonly applied to improve the expression level of a DNA or RNA sequence in a context that differs from that of the origin of the encoded sequence. It involves the adaptation of a nucleotide sequence to encode the intended amino acids, but by way of a nucleotide sequence that matches the codon preference (the tRNA repertoire) of the recombinant vector, the host cell, or the target organism in which the sequence will be expressed. Consequently, the nucleotide mutations applied are commonly silent. Such modifications are commonly planned in silico by using one of many available computer software programs, after which the desired nucleotide sequence can be prepared.

Therefore in an embodiment of the recombinant vector according to the invention, the recombinant protein is encoded by a nucleotide sequence that is codon optimised towards the type of the recombinant vector and/or to the species of the host cell or the target organism in which the recombinant vector will be applied. Preferably the type of the vector is a Mycoplasma bacterium, and the species of the host cell or the target is a ruminant.

Therefore in an embodiment of the recombinant vector according to the invention, the recombinant protein is encoded by a nucleotide sequence comprising the nucleotide sequence of one of SEQ ID NO: 36 through 40.

As is evident to the skilled person, these nucleotide sequences are represented here in DNA format and refer to the ‘coding strand’. For the complementary DNA strand, the ‘template’ strand, the sequence is the inverse-complement. Also, in an RNA format the T nucleotide would have to be replaced by an U. For use in another type of vector, e.g. another bacterium, virus, or host cell, some changes may be required. For example for expression by Mpneumo, these sequences were codon-optimised towards the codon use table of Mpneumo, as described in Weber et al. (2020, Molec. Syst. Biol., vol. 16, e9208). Specifically: in SEQ ID NO: 36 and 37 the codon TGA is used to encode tryptophan, as is common for some Mycoplasma's (see Inamine et al., 1990, J. of Bacter., vol 172, p. 504-506). However when used as universal genetic code TGA is a stop codon (Opal), and would have to be changed to TGG to encode tryptophan. Such adaptations are all within the routine capabilities of the skilled artisan.

The recombinant vector according to the invention may comprise a single nucleotide sequence encoding a recombinant protein for the invention, or may comprise more than one of such encoding nucleotide sequences for the invention. The multiple encoding nucleotide sequences may encode the same or different recombinant proteins for the invention. Alternatively the nucleotide sequence may encode more than one recombinant protein for the invention in a single sequence.

Therefore in an embodiment of the recombinant vector according to the invention, the recombinant vector comprises more than one encoding nucleotide sequence, each comprising a nucleotide sequence from one of SEQ ID NO: 36 through 40. In a preferred embodiment the recombinant vector comprises two encoding nucleotide sequences, either the nucleotide sequences of SEQ ID NO: 36 and SEQ ID NO: 37, or the nucleotide sequences of SEQ ID NO: 38 and SEQ ID NO: 39.

In another embodiment the recombinant vector comprises an encoding nucleotide sequence comprising more than one nucleotide sequence selected from SEQ ID NO: 36 through 40.

As indicated above, each of the nucleotide sequences comprised in a recombinant vector according to the invention, can encode further sequences such as e.g. marker sequences. In a preferred embodiment the nucleotide sequence comprises the amino acid sequence of a 6x His-tag or a Flag tag (SEQ ID NO: 7). More preferably the marker sequence is placed at one of the termini of the encoded recombinant protein for the invention; more preferably the marker sequence is placed at the C-terminus.

As described, the integration of an encoding nucleotide sequence in a recombinant vector according to the invention, in an expression cassette for the invention, can be done in different ways, orders or orientations, all using routine methods and materials.

As described, the recombinant vector according to the invention can advantageously be used to deliver and express a recombinant protein for the invention to a target animal, e.g. as a way to vaccinate that target. This may involve at some stage the introduction of that vector into a suitable host cell kept in vitro. Depending on the type of vector applied, that introduction into a host cell may require a carrier, some method of transfection, or may be guided by the vector itself. Nevertheless, once the vector is inside the host cell, the recombinant protein is expressed, and the host cell, being infected or transfected with the recombinant vector, can itself be used for the vaccination of a target.

Therefore in a further aspect the invention relates to a host cell comprising the recombinant vector according to the invention.

A “host cell” for the invention, is a eukaryotic- or a prokaryotic cell, which allows for the expression of the recombinant protein for the invention, from the recombinant vector according to the invention, after the introduction of that vector into said host cell, e.g. by way of transfection or infection.

A host cell according to the invention is typically manipulated and amplified in vitro, e.g. as cells in a culture that is a suspension (free or attached), or a cell-layer. When eukaryotic, the host cell can be a primary cell, prepared in vitro from a sample or tissue obtained from a human or animal. Typically primary cells can only perform a small and limited number of cell-divisions when in vitro. Alternatively the host cell can be an immortalised eukaryotic cell, for example from an established cell-line, which can grow and divide almost indefinitely. Depending on the type and species of the host cell, the expression of the recombinant protein for the invention may include some form of post-translational processing, such as e.g. signal peptide cleavage, disulphide bond formation, glycosylation, and/or lipid modification.

Examples of host cells for the invention are cells to be used for the expression of the recombinant protein(s) for the invention: e.g. bacterial cells (e.g. Mycoplasma bacteria) or eukaryotic cells (e.g. CHO cells, insect cells) transfected with a nucleic acid encoding the recombinant protein; or eukaryotic cells that can be infected with a recombinant virus or -bacterium that expresses the recombinant protein, etc.. All well-known in the art.

Also, the primary- or the immortalised host cell can be of the same- or from a different species as the target for a vaccine comprising the recombinant protein for the invention, or comprising the recombinant vector according to the invention.

The host cell for the invention is preferably an immortalised ruminant cell, e.g. an MDBK (Madin Darby Bovine kidney) cell.

In an embodiment of the host cell according to the invention, the host cell can be alive (i.e. replicative), or can be dead (e.g. after inactivation).

The recombinant vector and the host cell, both according to the invention can be constructed, cultured and used, with methods and materials well-known in the field of the invention.

Therefore in a further aspect the invention relates to a method for the manufacture of the composition according to the invention, the method comprising obtaining the recombinant protein or the combination of recombinant proteins from a vector or from a host cell both according to the invention.

As described, the skilled person has many ways at his disposal to implement the method according to the invention, varying from classic- to modern biotechnology, and any combination thereof. For example a recombinant protein for the invention can e.g. be produced synthetically, or can be isolated from a culture of recombinant bacteria or of host cells transformed or infected with a recombinant vector, all according to the invention. Alternatively the culture as a whole can be used, e.g. after inactivation, or may be used after limited purification such as centrifugation or filtration; all well-known in the art.

While a recombinant protein for the invention can be used in different ways, e.g. in detection or for diagnostics, its main advantageous use is in a vaccine against M. bovis and its consequences.

Therefore in a further aspect the invention relates to a vaccine for reducing infection or disease caused by M. bovis, the vaccine comprising the composition, the recombinant vector, and/or the host cell, all according to the invention, and a pharmaceutically acceptable carrier.

A “vaccine” is well-known to be a composition comprising an immunologically active compound, in a pharmaceutically acceptable carrier. The ‘immunologically active compound’, or ‘antigen’ is a molecule that is recognised by the immune system of the inoculated target and induces a protective immunological response from the humoral- and/or the cellular immune system of the target. For the present invention, the immunologically active compound is a recombinant protein for the invention, which can be provided, delivered, and/or expressed by the composition, the recombinant vector, and/or the host cell, all according to the invention.

In general a vaccine helps to reduce the infectious load or to shorten the duration of the replication of the pathogen against which the vaccination is directed.

The term “reducing” regards reducing in part or in whole the establishment or the proliferation of a productive infection by M. bovis, in organs of a susceptible target, or of the subsequent signs of disease. For the present invention, this is achieved for example by reducing the bacterial load or shortening the duration of M. bovis replication. In turn this leads to a reduction in the vaccinated target of the number, the intensity, or the severity of lesions and associated clinical signs of disease caused by the M. bovis infection.

The vaccine according to the invention is colloquially referred to as a vaccine ‘against M. bovis’ c.q. against M. bovis induced Mycoplasmosis, and provides for “reducing infection or disease” as may be caused by an M. bovis bacterium, such as symptoms of pneumonia, arthritis and/or mastitis.

Such reduction of infection or disease can readily be detected for instance by monitoring the immunological response following administration of the vaccine according to the invention, and by testing the appearance of clinical symptoms or mortality after an infection of vaccinated targets, e.g. by monitoring the targets' signs of disease, clinical scores, serological parameters, or by re-isolation of the infecting pathogen. For example by determining a reduction of the lung lesions caused by M. bovis infection, and a reduction of the colonisation of respiratory tissue. In animals these results can be compared to a response against a challenge infection in mock vaccinated animals. Several ways to assess M. bovis infection and symptoms of disease are known in the art.

In an embodiment, the vaccine according to the invention reduces the lung lesions caused by an infection with M. bovis in a susceptible target, by at least 10%, as compared to the level of lung lesions in a similar target, undergoing a similar infection, but not vaccinated with a vaccine according to the invention. The lung lesions are to be determined as total relative lung lesion scores according to Jericho & Langford (1982, Can. J. Comp. Med, vol. 46, p. 287-292). For example as described in the Examples: a reduction of the lung lesion score from 3.1 for mock vaccinated-challenged targets to 1.4 in vaccinated-challenged animals, is a reduction by 55%; similarly, a reduction of lung lesion scores from 3.1 to 0.7 is a reduction by 77%.

Preferably the vaccine according to the invention reduces M. bovis induced lung lesions by at least 20, 30, 40, 50, 60, 70, 75, or even by at least 77%, in this order of preference.

In an embodiment, the vaccine according to the invention reduces the colonisation of the respiratory tract of a susceptible target by an infection with M. bovis, by at least 10%, as compared to the level of colonisation in a similar target, undergoing a similar infection, but not vaccinated with a vaccine according to the invention. Colonisation is to be determined by performing and measuring trachea swabs, e.g. as described in the Examples. For example, a reduction of respiratory tract colonisation as measured by trachea swap from 73.847 CFU/ml in mock-vaccinated-challenged targets, to 2.550 CFU/ml in vaccinated-challenged animals, is a reduction by 96%; similarly, a reduction from 73.847 to 53 CFU/ml, is a reduction by 99%.

Preferably the vaccine according to the invention reduces M. bovis colonisation of the respiratory tract by at least 20, 30, 40, 50, 60, 70, 80, 90, 95, 96, 97, 98, or even by at least 99%, in this order of preference.

Further results of a vaccination according to the invention are seen in the restoration of the general health and well-being of the vaccinated targets. For example in ruminants, the protection against M. bovis also leads to increased economic performance which is reflected by parameters such as: reduced mortality, improved growth rate, better average daily weight gain, improved feed conversion, improved milk production, improved reproductive yields such as time between pregnancies and number- and health of offspring, and/or reduced costs for veterinary healthcare. Additionally, the increased health will allow a reduction in the use of antibiotics, which is an important goal for the agricultural community.

A “pharmaceutically acceptable carrier” is well-known to aid in the stabilisation and the administration of a vaccine, while being relatively harmless and well-tolerated by the target. Such a carrier can for instance be sterile water or a sterile physiological salt solution. In a more complex form the carrier can e.g. be a buffer, which can comprise further additives, such as a stabiliser or a preservant. Details and examples are for instance described in well-known handbooks such as: “Remington: the science and practice of pharmacy” (2000, Lippincott, USA, ISBN: 683306472), and: “Veterinary vaccinology” (P. Pastoret et al. ed., 1997, Elsevier, Amsterdam, ISBN 0444819681).

When the vaccine according to the invention comprises a recombinant vector that is a replicating virus, then the pharmaceutically acceptable carrier is preferably a composition stabilising that virus, or the host cell in which it is contained. Examples are several viral vaccine diluents, and stabilisers for frozen or freeze-dried storage, typically comprising e.g. a sugar, an amino acid, a physiological buffer (e.g. saline, PBS, or 50 mM HEPES), and often a bulky compound such as an albumin, a polymer etc.

Similarly, when the vaccine according to the invention comprises a recombinant vector according to the invention that is a nucleic acid or an RP, the pharmaceutically acceptable carrier can be a simple buffer, e.g. a phosphate buffer with 5% w/v sucrose.

Further, an additional carrier can be added to stabilise and/or deliver the recombinant vector according to the invention, e.g. to encapsulate the recombinant vector according to the invention that is a nucleic acid or an RP with an appropriate (nanoparticulate) carrier, such as a protein, polysaccharide, lipid or a polymer, as described. Preferably the additional carrier for a recombinant vector according to the invention that is an RP, comprises a nanogel that is a biodegradable polyacrylic polymer as described in WO 2012/165953.

As described, the recombinant vector or the host cell, both according to the invention, can be employed herein alive (i.e. replicative), or dead (non-replicative, or inactivated). In turn, a part of a composition comprising the recombinant vector or the host cell, both according to the invention, can be used for example as a: pellet, supernatant, concentrate, dialysate, extract, sonicate, lysate or fraction of a composition, e.g. a culture, comprising the vector and/or the host cell. All this is well-known to the skilled person.

The vaccine according to the invention is intended for administration to a target in need of such treatment. A “target” for the invention is any animal that is susceptible to infection by M. bovis, such as a ruminant.

The target can be of any weight, sex, or age at which it is susceptible to a vaccine according to the invention. However it is evidently favourable to treat healthy, uninfected targets, and to treat as early as possible to prevent any field infection and its consequences.

The vaccine according to the invention can thus be used either as a prophylactic- or as a therapeutic treatment, or both, as the recombinant protein it provides can induce an immune response that can interfere both with the establishment- and with the progression of an infection or disease caused by M. bovis.

Preferred target for the vaccine is a ruminant.

Therefore in an embodiment the vaccine according to the invention is for administration to a ruminant target.

For the invention, a “ruminant” regards any ruminant of relevance to veterinary science or to commercial farming operations. Preferably ruminant refers to bovine, caprine, ovine or cervine animals. More preferred are bovines, goats, and sheep. Most preferred ruminant is a bovine.

A “bovine” for the invention is preferably one selected from: taurine cattle (Bos taurus), zebu cattle (Bos indicus), buffalo, bison, yak, and wisent. More preferably the bovine is taurine- or zebu cattle.

The bovine can be of any type: dairy or beef, or parental stock for dairy- or beef type. Preferably the bovine is no more than 6 months old at its first vaccination with the vaccine according to the invention; more preferably no more than 3 months, 2 months, or even no more than 1 month old, in this order of preference.

In an embodiment the bovine is no more than 6, 5, 4, 3, 2, or even no more than 1 week old, in this order of preference.

The vaccine according to the invention can comprise additional immunoactive components. This can serve to enhance the immune protection already provided, or to expand it to other pathogens.

Therefore, in an embodiment, the vaccine according to the invention comprises at least one additional immunoactive component.

Such an “additional immunoactive component” may be an antigen, an immune enhancing substance, a cytokine, a further vaccine, or any combination thereof. This provides advantages in terms of cost, efficiency and welfare. Alternatively, the vaccine according to the invention, may itself be added to a vaccine.

A further advantageous effect of the reduction of the load of M. bovis in a target, by the vaccine according to the invention, is the reduction or even prevention of shedding of M. bovis by infected targets, and thereby of the spread of M. bovis, both vertically to offspring, and horizontally within a flock or population and within a geographical area. Consequently, the use of a vaccine according to the invention leads to a reduction of the prevalence of M. bovis.

Therefore further aspects of the invention are:
use of the vaccine according to the invention for reducing the prevalence of M. bovis in an animal, a population or a geographical area; and
the vaccine according to the invention for reducing the prevalence of M. bovis in an animal, a population or a geographical area.

The vaccine according to the invention is prepared by well-known methods, for example as described and exemplified herein, e.g. comprising the step of admixing of the recombinant vector for use according to the invention, or of the host cell for the invention, with the pharmaceutically acceptable carrier.

Therefore, in a further aspect, the invention relates to a method for the manufacture of the vaccine according to the invention, the method comprising the admixing of the composition, the recombinant vector, and/or the host cell, all according to the invention, with a pharmaceutically acceptable carrier.

Also various other compounds can be added to a vaccine according to the invention, e.g. stabilisers, carriers, adjuvants, diluents, emulsions, etc. Such additives are described in well-known handbooks such as: “Remington”, and “Veterinary Vaccinology” (both supra).

This way the efficacy of a vaccine according to the invention can be further optimised when needed by a skilled person, using routine techniques.

Therefore in an embodiment the vaccine according to the invention is characterised in that the vaccine comprises an adjuvant.

An “adjuvant” is a well-known vaccine ingredient that stimulates the immune response of a target in a non-specific manner. Many different adjuvants are known in the art. Examples of adjuvants are: complete- or incomplete Freund's adjuvant, vitamin E or alpha-tocopherol, non-ionic block polymers and polyamines such as dextran sulphate, Carbopol™, pyran, Saponin, such as: Quil A™, or Q-vac™. Saponin and vaccine components may be combined in an ISCOM™.

Furthermore, peptides such as muramyl dipeptides, dimethylglycine, tuftsin, are often used as adjuvant, and mineral oil e.g. Bayol™, Drakeol™, Klearol™, or Marcol™, Montanide™ or light mineral (paraffin) oil; non-mineral oil such as squalene, squalane; vegetable oils or derivatives thereof, e.g. ethyl-oleate. Also combination products such as ISA™ (Seppic), or DiluvacForte™ and Xsolve™ (both MSD Animal Health) can advantageously be used. A further option is the use of SVEA adjuvant (comprising squalane and vitamin E-acetate) as disclosed in WO 2018/115435.

A handbook on adjuvants and their uses and effects is: “Vaccine adjuvants” (Methods in molecular medicine, vol. 42, D. O'Hagan ed., 2000, Humana press, NJ, ISBN: 0896037355).

In an embodiment of the vaccine according to the invention the adjuvant is one or more selected from an aluminium-salt and an oil. The oil is a mineral oil, or is a non-mineral oil; preferably the oil is a mineral oil. The aluminium salt is preferably an aluminium-hydroxide. Much used aluminium salt is aluminium-hydroxide, for example as: Alhydrogel™ (Brenntag Biosector), Rehydragel™ (Reheis), or Rehsorptar™ (Armour Pharmaceutical).

Much used mineral oil adjuvant in veterinary vaccines is a light (or white) liquid paraffin oil, such as Drakeol® 6VR (Penreco), Marcol® 52 (Exxon Mobile), and Klearol® (Sonneborn). Alternative is a premixed mineral oil/emulsifier mixture such as the Montanide® range from Seppic, France. Common non-mineral oil adjuvants are squalene and squalane (shark liver oil), ethyl oleate, and tocopherol (Vitamin E).

The oil phase may contain excipients such as an emulsifier and stabilisers. Common emulsifiers for vaccines are sorbitan monooleate (Span® 80) and polyoxyethylene-sorbitan-monooleate (polysorbate 80, or Tween® 80). Common emulsion-stabilisers are benzyl alcohol, and triethanolamine.

The vaccine with an oily adjuvant can be formulated as an emulsion of a watery- and an oily phase; preferably the emulsion is of a type selected from: water-in-oil (w/o), oil-in-water (o/w), water-in-oil-in-water (w/o/w), and double oil-emulsion (o/w/o).

More preferred is a vaccine according to the invention that is adjuvated with an oil, and is formulated as a water-in-oil emulsion.

Therefore, in an embodiment of the vaccine according to the invention, the vaccine is formulated as a water-in-oil emulsion.

In a preferred embodiment of the vaccine according to the invention comprising an adjuvant, the adjuvant comprises an oil; preferably the oil is a light paraffin oil.

More preferably the adjuvant also comprises an aluminium salt; the aluminium salt is preferably an aluminium-hydroxide.

In an embodiment of the vaccine according to the invention, the vaccine is an aqueous formulation with aluminium hydroxide and a saponin; preferably having 1.7% w/v of aluminium hydroxide, and/or preferably having 0.03 v/v % saponin.

In a further aspect, the invention relates to a method for the manufacture of the vaccine according to the invention, the method comprising the admixing of the composition, the recombinant vector, and/or the host cell, all according to the invention, with an adjuvant.

General techniques and considerations that apply to the manufacture of vaccines under well-known standards for pharmaceutical production are described for instance in governmental directives and regulations (Pharmacopoeia, 9CFR) and in well-known handbooks (“Veterinary vaccinology” and: “Remington”, both supra). Commonly such vaccines are prepared sterile, and are prepared using excipients of pharmaceutical quality grade.

Such preparations will incorporate microbiological tests for sterility, and absence of extraneous agents, and may include studies in vivo or in vitro for confirming efficacy and safety. After completion of the testing for quality, quantity, sterility, safety and efficacy, the vaccine can be released for sale. All these are well-known to a skilled person.

Therefore in a further aspect the invention relates to a use of the composition, of the recombinant vector, and/or of the host cell, all according to the invention, for the manufacture of a vaccine for reducing infection or disease caused by M. bovis.

Depending on the route of application of the vaccine according to the invention, it may be necessary to adapt the vaccine's composition. This is well within the capabilities of a skilled person, and generally involves the fine-tuning of the efficacy or the safety of the vaccine. This can be done by adapting the vaccine dose, quantity, frequency, route, by using the vaccine in another form or formulation, or by adapting the other constituents of the vaccine (e.g. a stabiliser or an adjuvant).

Preferably a vaccine according to the invention is formulated as an injectable liquid, suitable for injection either intradermal or parenteral. The injectable liquid can e.g. be a suspension, solution, dispersion, or emulsion.

In an embodiment, the vaccine according to the invention is for administration by parenteral route. Preferably by intramuscular- or subcutaneous route.

In an embodiment, the vaccine according to the invention is for administration by mucosal route, e.g. by oral, nasal, or ocular route.

Determination of what is an immunologically effective amount of the recombinant protein for the invention in the vaccine according to the invention, or the optimisation of the vaccine's volume per dose, are both well within the capabilities of the skilled artisan.

In an embodiment of the vaccine according to the invention, the vaccine comprises from 0.1 μg to 10 mg per dose of a recombinant protein for the invention.

As the skilled person will appreciate, the amount of total protein per vaccine dose will also be determined by the relative purity of the vaccine to be used: as a purified subunit protein much less total protein will be included in a vaccine dose, as compared to the use of an inactivated culture of a virus- or bacterial vector or of a host cell., etc. All this is well-known to a skilled person.

The volume per dose of the vaccine according to the invention can be selected according to the characteristics of the specific vaccine applied, the characteristics of the target, and the intended route of administration. Parenteral injection is commonly done with a dose of 0.01-10 ml/target. Fora bovine, the preferred volume per dose is 0.5 ml for subcutaneous route, and 1-2 ml for i.m. route.

In a further aspect the invention relates to the composition, the recombinant vector, and/or the host cell, all according to the invention, for use as a vaccine for reducing infection or disease caused by M. bovis.

Preferably, said use as a vaccine for the invention incorporates all embodiments and preferences of the composition, the recombinant vector, and/or the host cell, all according to the invention, as described herein above.

In a further aspect the invention relates to a method for reducing infection or disease caused by M. bovis in a target, the method comprising administering to said target the vaccine according to the invention.

Preferably the method for reducing according to the invention incorporates all embodiments and preferences of the vaccine according to the invention, as described herein above.

The dosing regimen for applying the vaccine according to the invention to a target organism can be in single or multiple doses, in a manner compatible with the formulation of the vaccine, and in such an amount as will be immunologically effective.

Preferably, the regimen for administering a vaccine according to the invention is integrated into existing vaccination schedules of other vaccines that the target may require, in order to reduce stress to the target and to reduce labour costs. These other vaccines can be administered in a simultaneous, concurrent or sequential fashion, in a manner compatible with their licensed use.

Preferably the first administration of a vaccine according to the invention to a target only needs to be given once, a so-called single-shot vaccine. This does not rule out that a target may require optional yearly booster vaccinations.

In an embodiment of the use as a vaccine for the invention, and of the method for reducing according to the invention, the use and the method comprise an administration regimen wherein for the first administration of a vaccine according to the invention to a target a priming- and a booster dose is administered, optionally followed by a yearly re-administration.

The prime and the booster vaccination will typically be given between 1 and 12 weeks apart, preferable between 2 and 10, 3 and 8, or even between 4 and 6 weeks apart.

The invention will now be further described with reference to the following, non-limiting, examples.

### LEGEND TO THE FIGURES

FIG. 1: Results from vaccination-challenge experiment testing different compositions with recombinant proteins according to the invention. Displayed on the vertical axis are the relative lung lesion scores per animal. The short horizontal bars indicate the median value found for each group. Details are described in Example 2.

### EXAMPLES

### Example 1: Preparation of Recombinant Proteins of M. bovis Epitopes

Recombinant proteins comprising the epitopes as defined for the invention were constructed, whereby the epitopes to be expressed were grouped in a few sets as follows (epitope numbers as indicated in Tables 3 and 4 above):
Set 1: epitope numbers 1-10 (SEQ ID NO: 8-17),
Set 2: epitope numbers 11-18 (SEQ ID NO: 18-25), and
Set 3: epitope numbers Vsp1-Vsp5 (SEQ ID NO: 26-30).

In the nucleotide sequence constructs encoding the epitope-containing recombinant protein(s) for the invention, these epitope sequences were interspersed with linker sequences Link1 through LinkS (SEQ ID NO: 2-6), in an alternating way as described herein.

Further, the epitope sets 1 and 2 were each prepared in two ways numbered a and b, whereby the order of the epitopes was mixed-up; thus resulting in sets 1a, 1b and 2a, 2b respectively. These are the encoding nucleotide sequence constructs described in SEQ ID NO: 36-40, encoding the recombinant proteins SEQ ID NO: 31-35, respectively.

These sets of epitopes were then expressed by a recombinant vector, specifically an M. pneumoniae (Mpneumo) bacterium. For the expression by the Mpneumo vector each construct was given a signal sequence to target expression to the bacterial surface. The signal sequence was put at some distance by two more copies of Linker 1 (SEQ ID NO: 2). Also, to be able to detect expression and isolate the recombinant proteins, the different constructs were provided with a nucleotide sequence encoding a marker sequence, to be expressed at their C-terminus. Constructs 1a, 2a and 3 were provide with a Flag-tag (SEQ ID NO: 7), and constructs 1b and 2b, with a 6x His-tag.

This resulted in constructs encoding recombinant proteins for the invention, with the following schematic lay-outs:

Construct 1a:
Signal-3xlink1-Ep1-link2-Ep2-link3-Ep3-link4-Ep4-link5-Ep5-link1-Ep7-link2-Ep6-link3-Ep8-link4-Ep9-link5- Ep10-link1-FlagTag

Construct 1b:
Signal-3xlink1-Ep5-link2-Ep7-link3-Ep6-link4-Ep9-link5-Ep10-link1-Ep8-link2-Ep1-link3-Ep2-link4-Ep4-link5-Ep3-link1-HisTag

Construct 2a:
Signal-3xlink1-Ep11-link2-Ep12-link3-Ep12-link4-Ep14-link5-Ep15-link1-Ep16-link2-Ep17-link3-Ep18-link1-FlagTag

Construct 2b:
Signal-3xlink1-Ep17-link2-Ep18-link3-Ep14-link4-Ep15-link5-Ep16-link1-Ep13-link2-Ep12-link3-Ep11-link1-HisTag

Construct 3:
Signal-3xlink1-Vsp4-link2-Vsp5-link3-Vsp2-link4-Vsp3-link5-Vsp1-link1-FlagTag

The nucleic acids encoding the various constructs for the invention were designed and codon optimized towards the codon usage table of Mpneumo (as described by Weber et al., 2020, supra) and prepared by a commercial supplier. The sequences were verified, and the synthetic genes were inserted in a pMTn series transposon cloning plasmid; the plasmids were electroporated into Mpneumo resulting in the random transposon insertion of the epitope constructs into its genome, essentially as described in Pich et al. (2006, Microbiology, vol. 152, p. 519-527).

The encoding nucleotide sequences for these constructs were inserted into the Mpneumo vector in combinations: 1a and 1b in one; 2a and 2b in another, and 3 in yet another Mpneumo bacterial expression vector.

The promoter used to drive the expression of the double- or single epitope-construct inserts was a 22 nucleotide section from the putative Mg438 gene of M. genitalium, as described in Pich et al., 2006 (supra).

The resulting recombinant Mpneumo bacteria were then cultured in a growth medium resembling Friis medium with 10% porcine serum added, and passaged once in the same medium for expansion before harvest. Expression of the fusion constructs was checked by Western blot, by detecting the Flag- or His-tag respectively using monoclonal antibodies.

The bacteria were inactivated with binary ethylenimine (BEI) at 0.8% v/v concentration for 24 hours at 37° C., with constant shaking. This was repeated once more. Next the BEI was neutralised with thiosulfate by incubation for 24 hours at 37° C. with constant shaking.

The inactivated bacteria were quantified by protein content using a BCA assay. Next the mixture was formulated with the described aluminium-hydroxide—saponin adjuvant, by first mixing the antigen with aluminium hydroxide under constant stirring at room temperature, followed by the addition of a solution containing saponin, so that the final composition had 1.7% w/v of aluminium hydroxide, and 0.03 v/v % saponin. After complete mixing, the pH was adjusted to 7.3. This adjuvated vaccine was administered to calves.

### Example 2: Vaccination-Challenge Experiment with Recombinant Proteins Comprising M. bovis Epitopes

2.1. Introduction

This experiment was done to determine the safety and efficacy of an M. bovis vaccine based on recombinant proteins comprising epitopes from specific M. bovis proteins. The main way to determine efficacy of protection against an M. bovis challenge, is by scoring the lung lesions induced and the bacterial load of challenge bacteria in the trachea. As vector for expression of the recombinant proteins for the invention served Mpneumo bacteria, which were inactivated and mixed with adjuvant prior to vaccination, as described in Example 1.

2.2. Material and Methods

2.2.1. Experimental Set-Up

Fifteen calves were included in this study, divided over 3 groups of 5 animals. Two groups received different mixtures of the inactivated Mpneumo bacteria expressing the recombinant proteins for the invention with M. bovis epitopes. These were formulated with aluminium-hydroxide—saponin adjuvant. Mix 1 contained 200 μg of each of 2 different inactivated bacterial cultures, one culture expressing constructs 1a and 1b, and one culture expressing constructs 2a and 2b, as described in Example 1. Mix 2 had the same compounds as in mix 1, but in addition also had 200 μg of bacteria expressing construct 3. A negative control group was also included, that group received a mock vaccine of PBS in adjuvant.

All calves were vaccinated according to a prime-boost schedule with a 3 week interval, and received a 2 ml dose given intra-muscularly in the neck. Blood samples were taken prior to vaccination, throughout the study, and at necropsy after euthanisation, to allow serology testing. Additionally after vaccination, health assessments were done, to judge the systemic response of the calves and the occurrence of local reactions.

Two weeks after boost, the calves were given a challenge infection using virulent M. bovis, on three consecutive days. Necropsy was performed at about two weeks after challenge.

2.2.2.Treatments of Experimental Animals

Holstein-Frisian calves of 4-6 weeks old, identified individually by ear tag, were assembled on one farm, and given a primo vaccination, and a booster vaccination 3 weeks later. After two more weeks they were transported to containment facilities, where they were given the challenge inoculations. At 16 days after the last challenge inoculation, the calves were sedated, euthanised and necropsied.

The calves were given examinations for general health by a veterinarian, both before vaccination and before challenge, and only calves that were clinically healthy at start, were used in the experiment. The calves were fed standard rations, and water was available ad libitum. Checks for any anti-M. bovis antibodies prior to vaccination was done on blood samples taken 1 week before the primo vaccination, which were tested using the BIO K™260—Monoscreen AbELISA Mycoplasma bovis kit (BIO-X Diagnostics S.A.).

Division over the groups was such that the average age in each group was the same.

Before- and 3 days after each vaccination, animals were palpated at the injection site to determine any local reactions. Also rectal temperature readings were taken before and after vaccinations.

Blood samples (serum and heparin) were taken from the jugular vein, on the day of- and prior to the vaccinations, at day of first challenge inoculation, and at the end of the experiment.

At necropsy, percentage lung lesion score was recorded for each calf individually using the procedure described by Jericho & Langford (supra). Also two samples were taken from the lungs for histology, one from the left- and one from the right cranial lobe. Tracheal swabs were taken from an area of 1 cm² just before the bifurcation point, using a mould for consistent sampling areas between animals. The samples were taken by single swipe. Further, the right cranial lung lobe of animals from group 1 was flushed using 10 ml SP4 medium with 100 μg/ml ampicillin.

2.2.3. Challenge

200 μl of M. bovis strain JF4278, low passage stock, was inoculated into 20 ml of SP4 medium with 100 μg/ml ampicillin, and incubated at 37° C. for 24 hours. Subsequently 8 ml of the overnight culture was subcultured into 800 ml SP4 medium with 800 μg/ml ampicillin, and incubated at 37° C. for 24 hours. The challenge material was plated on Mycoplasma agar (Mycoplasma Experience™) plates, for quantification and to test the viability and absence of contaminations. Plates were incubated at 37° C. in 5% CO₂ for five days.

All animals were challenged intratracheally once daily on three consecutive days, by way of a tube via the nose, and reaching to the bifurcation of the trachea. For each inoculation 30 ml challenge culture was administered having about 10\hat{ }9 M. bovis JF4278 cells/ml.

2.2.4.Laboratory Procedures

Plain blood- and heparin blood samples were taken to determine the reaction of the animals to the vaccines. After euthanising the animals, several samples and swabs were taken during necropsy. Lung lesion scores were assessed and compared between the groups. Nasal and tracheal swabs were taken as well during necropsy, to measure the M. bovis load in the animals. These results were used to determine if there was a good “vaccine take”, to check if there was no co-infection, and to determine the efficacy of the vaccines.

2.3. Results

Vaccine-take was confirmed by the detection of antibodies against the vector bacteria, in blood samples from the experimental animals. The animals in group 1 (PBS vaccine) showed only background serological response against the Mpneumo vector, with an average Log2 titre of 7.6 at day of challenge. However both vector-vaccinated groups 2 and 3 showed a significant anti-vector response, as both groups had an average antibody titre of 12 Log2, at day of challenge.

This demonstrates that all the vaccinated groups had indeed received the bacterial vector-expressed antigen, and this had mounted an immune response.

After the vaccinations, no clear signs of vaccination-reactions, local or systemic, such as swelling, fever, malaise, etc., were observed.

Trachea Swabs

The trachea swabs taken at 16 days post challenge were plated on Mycoplasma agar and quantified, and results are presented in Table 5. Numbers given are the averages per group in CFU/ml, with their standard deviation (SD).

As is clear from these results, the vaccination as applied to Group 2 already strongly reduced the colonisation by the M. bovis challenge bacteria by 96%. The vaccine of Group 3 was even more effective, and reduced colonisation by 99%.

TABLE 5
Results of challenge bacteria re-isolation

CFU/ml

| Group | Average | SD |
|---|---|---|
| 1 - PBS | 73847 | 152365 |
| 2 - constr. 1a-1b and 2a-2b | 2550 | 625 |
| 3 - constr. 1a-1b, 2a-2b, and 3 | 53 | 74 |

Lung Lesion Scores

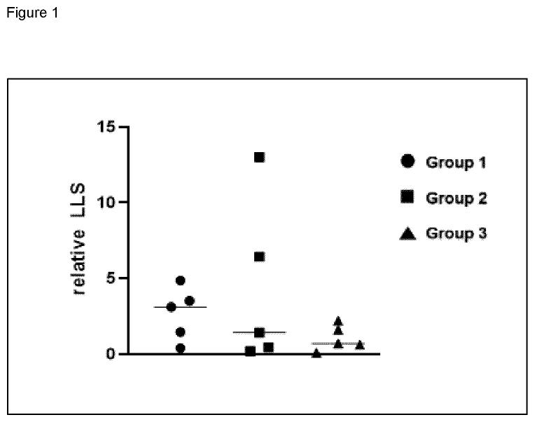

The total relative lung lesion scores (LLS) at 16 days after challenge were determined as described. Results are presented in FIG. 1. Because of the nature of these experiments, some variability in the individual scores is inevitable. Therefore it is best to focus on the median LLS per group; these were:
Group 1 (PBS): 3.1
Group 2 (mix of 1a, 1b, 2a, and 2b): 1.4
Group 3 (mix of 1a, 1b, 2a, 2b, and 3): 0.7

These results present the same trend as was found for the trachea swabs: the vaccine administered to Group 2 (the mixture of recombinant proteins 1a-1b and 2a-2b) was already capable of strongly reducing the lung lesions induced by challenge infection by 55%, as compared to the mock vaccinated-challenged calves. Remarkably, the vaccine administered to Group 3 (the mixture of recombinant proteins 1a-1b, 2a-2b, and 3) was even more effective, and reduced LLS by 77%. Also this reduced the spread among the group 3 calves.

Such strong reduction of lung lesions scores, after a severe M. bovis challenge infection, has not been reported before.

2.4. Conclusions

No indications of vaccine-enhanced disease were observed in the vaccinated groups, nor any local reactions or undesired systemic response. This indicated that the vaccines of the invention are safe.

Group 2 and in particular group 3 had clearly lower lung lesion scores, than did the mock-vaccinated-challenged group 1. In addition, group 2 and in particular group 3 also had lower average loads of challenge bacteria in the trachea, than the calves of group 1 had.

Taken together these findings strongly support the safety and the efficacy of the vaccines according to the invention, against infection and disease caused by M. bovis infection.

1. Composition comprising one or more recombinant proteins, characterised in that the recombinant protein or the combination of recombinant protein comprises at least one epitope from each of the Mycoplasma bovis (M. bovis) proteins with the GenBank accession number: WP_014829937, WP_075271052, WP_013456547, SBO45938, WP_013455936, WP_075271207, WP_013954974, WP_013954588, WP_013456028, WP_013954511, WP_075271115, WP_013456252, and WP_041309176, or from a homologue of said M. bovis proteins.

2. The composition according to claim 1, characterised in that the recombinant protein or the combination of recombinant proteins also comprise at least one epitope from each of the M. bovis Vsp proteins with the GenBank accession number: SBO46569, SBO46572, SBO46576, and SBO46580, or from a homologue of said M. bovis Vsp proteins.

3. The composition according to claim 1, characterised in that the recombinant protein or the combination of recombinant proteins also comprise one or more additional sequences selected from: signal-, transmembrane-, anchor-, linker-, spacer-, marker-, and cleavage sequences.

4. The composition according to claim 1, characterised in that the recombinant protein or the combination of recombinant proteins comprise each of the epitopes from SEQ ID NO: 8 through 25, or a homologue of said epitopes.

5. The composition according to claim 4, characterised in that the recombinant protein or the combination of recombinant proteins also comprise each of the epitopes from SEQ ID NO: 26 through 30, or a homologue of said epitopes.

6. Recombinant vector capable of expressing the recombinant protein or the combination of recombinant proteins as defined in claim 1, and wherein said vector is selected from a nucleic acid, a replicon particle (RP), a virus, and a bacterium.

7. The recombinant vector according to claim 6, characterised in that:
a. the nucleic acid is a DNA expression plasmid or an RNA molecule;
b. the RP is an Alphavirus RP;
c. the virus is selected from the group consisting of: a Herpesvirus, a Poxvirus, a d. Retrovirus, a Paramyxovirus, a Rhabdovirus, a Baculovirus and an Adenovirus; or
d. the bacterium is selected from the group consisting of the genera: Escherichia, Bacillus, Salmonella, Caulobacter, Lactobacillus, and Mycoplasma.

8. Host cell comprising the recombinant vector according to claim 6.

9. A method for the manufacture of a composition comprising one or more recombinant proteins, the method comprising obtaining the recombinant protein or the combination of recombinant proteins from a vector according to claim 6 wherein the recombinant protein or the combination of recombinant protein comprises at least one epitope from each of the Mycoplasma bovis (M. bovis) proteins with the GenBank accession number: WP_014829937, WP_075271052, WP_013456547, SBO45938, WP_013455936, WP_075271207, WP_013954974, WP_013954588, WP_013456028, WP_013954511, WP_075271115, WP_013456252, and WP_041309176, or from a homologue of said M. bovis proteins.

10. A vaccine for reducing infection or disease caused by M. bovis, the vaccine comprising the composition according to claim 1, and a pharmaceutically acceptable carrier.

11. The vaccine according to claim 10 characterised in that the vaccine comprises an adjuvant.

12. A Method for the manufacture of a vaccine, the method comprising the admixing of the composition according to claim 1, with a pharmaceutically acceptable carrier.

13. (canceled)

14. (canceled)

15. A Method for reducing infection or disease caused by M. bovis in a target, the method comprising administering to said target the vaccine according to claim 10.
